(19) United States
(12) Patent Application Publication
(10) Pub. No.: US 2024/0000530 A1
(43) Pub. Date: Jan. 4, 2024

(54) ROBOTIC AND MANUAL ASPIRATION CATHETERS

(71) Applicant: Auris Health, Inc., Redwood City, CA (US)

(72) Inventors: Russell W. PONG, Newark, CA (US); Thomas R. JENKINS, Alameda, CA (US); Luis Andrade BAEZ, JR., Mountain View, CA (US); Bryan D. KNODEL, Flagstaff, AZ (US); Ka Chun WONG, South San Francisco, CA (US); Ken VIEN, San Jose, CA (US)

(21) Appl. No.: 18/324,842

(22) Filed: May 26, 2023

Related U.S. Application Data
(63) Continuation of application No. PCT/IB2021/062087, filed on Dec. 21, 2021.
(60) Provisional application No. 63/132,864, filed on Dec. 31, 2020.

(51) Int. Cl. A61B 34/37; A61B 17/22; A61B 90/96; A61B 34/00; A61B 90/98

(52) U.S. Cl. CPC A61B 34/37; A61B 17/22; A61B 90/96; A61B 34/74; A61B 90/98; A61B 2017/22079; A61B 2034/715; A61B 2034/301

(57) ABSTRACT

An aspiration catheter can include an elongate shaft and an instrument base coupled to the shaft and configured to control actuation of at least a distal portion of the shaft. The shaft can include a lumen configured to couple to an aspiration system to provide aspiration to a target site, such as to remove an object from a patient. The instrument base can be controlled robotically and/or manually to articulate at least the distal portion of the shaft.

### RELATED APPLICATION(S)

This application is a continuation of International Application No. PCT/IB2021/062087, filed Dec. 21, 2021, and entitled ROBOTIC AND MANUAL ASPIRATION CATHETERS, which claims priority to U.S. Provisional Application No. 63/132,864, filed Dec. 31, 2020, and entitled ROBOTIC AND MANUAL ASPIRATION CATHETERS, the disclosures of which are hereby incorporated by reference in their entirety.

### BACKGROUND

Various medical procedures involve the use of one or more medical devices for accessing a target anatomical site in a patient. In some instances, the improper use of certain devices when accessing the site in connection with a procedure can adversely affect the health of the patient, the integrity of the medical device(s), and/or the efficacy of the procedure.

### SUMMARY

In some implementations, the present disclosure relates to a robotically-controllable catheter assembly comprising an elongate shaft including a lumen and configured to couple to an aspiration system to provide aspiration to a target site via the lumen, and an instrument base coupled to the elongate shaft and configured to control actuation of the elongate shaft. The instrument base includes a drive input assembly configured to couple to a drive output assembly associated with a robotic arm.

In some embodiments, the elongate shaft includes another lumen, and the robotically-controllable catheter assembly further comprises an elongate movement member slidably disposed in the other lumen and connected to a distal end of the elongate shaft. The drive input assembly can be connected to the elongate movement member to control articulation of the elongate shaft.

In some embodiments, the instrument base includes a port coupled to a proximal end of the elongate shaft and configured to couple to the aspiration system. Further, in some embodiments, the instrument base includes an identification element associated with an identifier for the robotically-controllable catheter assembly. The identification element can include at least one of a radio-frequency identification tag, a Quick Response (QR) code, a bar code, or a magnet.

In some embodiments, the robotically-controllable catheter assembly further comprises a handheld instrument adapter configured to receive manual input to control manipulation of the elongate shaft. The handheld instrument adapter can include a coupler configured to couple to the drive input assembly of the instrument base and a manual actuator connected to the coupler and configured to manipulate the coupler. In examples, the coupler includes a gear assembly engaged with the manual actuator and configured to engage with the drive input assembly. Further, in examples, the robotically-controllable catheter assembly further comprises a pull wire configured to manipulate the elongate shaft. The coupler can include a tensioning mechanism configured to disengage the manual actuator from manipulating the drive input assembly and configured to adjust tension of the pull wire.

In some implementations, the present disclosure relates to a manually-controllable catheter comprising an elongate shaft including a lumen and configured to couple to an aspiration system to provide aspiration to a target site via the lumen; and an instrument handle coupled to the elongate shaft and including a manual actuator configured to control actuation of the elongate shaft.

In some embodiments, the elongate shaft includes a wire lumen, and the manually-controllable catheter further comprises a pull wire slidably disposed in the wire lumen and connected to a distal end of the elongate shaft. The manual actuator can be connected to the pull wire to control articulation of the elongate shaft. Further, in some embodiments, the instrument handle includes a port coupled to a proximal end of the elongate shaft and configured to couple to the aspiration system.

In some embodiments, the manual actuator is configured to be actuated by a thumb of a user when the instrument handle is held by the user in an overhand manner. Further, in some embodiments, the manual actuator is configured to be actuated by a thumb of a user when the instrument handle is held by the user in an underhand manner.

In some implementations, the present disclosure relates to a system comprising a base, a coupler rotatably supported in the base, and a first manual actuator operatively coupled to the coupler. The coupler is configured to couple to a drive input assembly of a robotically-controllable medical instrument. The first manual actuator is configured to manipulate the coupler to cause the robotically-controllable medical instrument to articulate.

In some embodiments, the coupler includes an engagement assembly coupled to the first manual actuator and configured to couple to the drive input assembly of the robotically-controllable medical instrument. In examples, the engagement assembly includes (i) a first engagement member to engage with the manual actuator, (ii) a second engagement member configured to engage with the drive input assembly, and (iii) a disengagement mechanism configured to disengage a coupling of the first engagement member to the second engagement member. Further, in examples, the disengagement mechanism includes a second manual actuator configured to receive manual input to disengage the coupling of the first engagement member to the second engagement member.

In some embodiments, the system further comprises the robotically-controllable medical instrument including (i) an elongate shaft configured to couple to an aspiration system to provide aspiration to a target site, and (ii) an instrument base coupled to the elongate shaft and configured to control actuation of the elongate shaft. The instrument base can include the drive input assembly. In examples, the elongate shaft includes a lumen, and the robotically-controllable medical instrument further includes an elongate movement member slidably disposed in the lumen and connected to a distal end of the elongate shaft. The drive input assembly can be connected to the elongate movement member to control articulation of the elongate shaft. Further, in examples, the coupler includes a tensioning mechanism configured to disengage the first manual actuator from manipulating the drive input assembly and configured to adjust tension of the elongate movement member. Moreover, in examples, the instrument base includes a port coupled to a proximal end of the elongate shaft and configured to couple to the aspiration system.

In some embodiments, the coupler includes a gear assembly engaged with the first manual actuator and configured to engage with the drive input assembly.

In some implementations, the present disclosure relates to a system comprising an elongate shaft and a handle coupled to the elongate shaft. The elongate shaft includes a distal end portion, a proximal end portion, and a lumen. The elongate shaft is configured to couple to an aspiration system to provide aspiration via the lumen. The handle is configured to operate in: a robotic mode in which the handle receives robotic input to control articulation of the elongate shaft, and a manual mode in which the handle receives manual input to control articulation of the elongate shaft.

In some embodiments, the system further comprises a robotic arm including a drive output assembly configured to provide the robotic input to the handle. The handle can be coupled to the drive output assembly of the robotic arm. Further, in some embodiments, the handle includes a manual actuator coupled to the elongate shaft and configured to receive the manual input.

In some embodiments, the handle includes an instrument base configured to receive the robotic input and an adapter configured to couple to the instrument base. The adapter can include a manual actuator configured to receive the manual input. In examples, the adapter includes a coupler configured to couple to a drive input assembly of the instrument base. The coupler can include (i) a first engagement member to engage with the manual actuator, (ii) a second engagement member configured to engage with the drive input assembly, and (iii) a disengagement mechanism configured to disengage a coupling of the first engagement member to the second engagement member. Further, in examples, the disengagement mechanism includes another manual actuator configured to receive manual input to disengage the coupling of the first engagement member to the second engagement member.

In some embodiments, the elongate shaft includes a pull wire configured to manipulate the distal end portion of the elongate shaft. In examples, the handle includes a tensioning mechanism configured to adjust a tension of the pull wire.

In some embodiments, the handle includes a port configured to connect to the lumen and the aspiration system.

For purposes of summarizing the disclosure, certain aspects, advantages and features are described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the disclosure. In addition, various features of different disclosed embodiments can be combined to form additional embodiments, which are part of this disclosure. Throughout the drawings, reference numbers may be reused to indicate correspondence between reference elements.

FIG. 1 illustrates an example robotic medical system arranged for a diagnostic and/or therapeutic ureteroscopy procedure in accordance with one or more embodiments.

FIG. 2 illustrates an example robotic medical system arranged for a diagnostic and/or therapeutic bronchoscopy procedure in accordance with one or more embodiments.

FIG. 3 illustrates an example table-based robotic system in accordance with one or more embodiments.

FIG. 4 illustrates example medical system components that may be implemented in any of the medical systems of FIGS. 1-3 in accordance with one or more embodiments.

FIG. 5 illustrates an example catheter disposed in the kidney of a patient in accordance with one or more embodiments.

FIG. 6 illustrates an example catheter including a shaft and a handle in accordance with one or more embodiments.

FIG. 7A illustrates a side view of the shaft of the catheter from FIG. 6 in accordance with one or more embodiments.

FIG. 7B illustrates a cross-sectional view of the shaft of the catheter from FIG. 6 in accordance with one or more embodiments.

FIG. 8A illustrates a perspective view of an example robotically-controllable catheter in accordance with one or more embodiments.

FIG. 8B illustrates a bottom view of the example robotically-controllable catheter from FIG. 8A in accordance with one or more embodiments.

FIG. 8C illustrates a perspective view of a bottom of the example robotically-controllable catheter from FIGS. 8A-8B in accordance with one or more embodiments.

FIG. 9-1 illustrates a top view of the instrument base of the catheter from FIGS. 8A-8C in accordance with one or more embodiments.

FIG. 9-2 illustrates a top view of the instrument base of the catheter from FIGS. 8A-8C with a top portion of the instrument base removed in accordance with one or more embodiments.

FIG. 10 illustrates example components of the instrument base of the catheter from FIGS. 8A-8C in accordance with one or more embodiments.

FIG. 11 illustrates an exploded view of an example instrument device manipulator assembly associated with a robotic arm in accordance with one or more embodiments.

FIG. 12-1 illustrates an example manual adapter configured to couple to a robotically-controllable medical instrument in accordance with one or more embodiments.

FIG. 12-2 illustrates example components of the adapter from FIG. 18-1 in an exploded view in accordance with one or more embodiments.

FIG. 12-3 illustrates an example engagement assembly engaged with a manual actuator of the adapter from FIG. 12-1 in accordance with one or more embodiments.

FIG. 12-4 illustrates the engagement assembly from FIG. 12-3 in an exploded view in accordance with one or more embodiments.

FIG. 12-5 illustrates a bottom view of an example manual actuator and gear/coupler of the adapter from FIG. 12-1 in accordance with one or more embodiments.

FIGS. 13A and 13B illustrate the adapter from FIG. 12-1 coupled to a robotically-controllable catheter in accordance with one or more embodiments.

FIGS. 14A, 14B, and 14C illustrate perspective, side, and top views, respectively, of an example manually-controllable catheter in accordance with one or more embodiments.

FIGS. 15A, 15B, and 15C illustrate side and perspective views of the example manually-controllable catheter from FIGS. 14A-14C in accordance with one or more embodiments.

FIG. 16 illustrates the manual actuator and other features of the example manually-controllable catheter from FIGS. 14A-14C in accordance with one or more embodiments.

FIG. 17 illustrates the example manually-controllable catheter of FIGS. 14A-14C held by a user in accordance with one or more embodiments.

FIG. 18-1 illustrates another example manually-controllable catheter in accordance with one or more embodiments.

FIG. 18-2 illustrates example internal components of the manually-controllable catheter of FIG. 18-1 in accordance with one or more embodiments.

FIG. 19 illustrates the example manually-controllable catheter of FIGS. 18-1 and 18-2 held by a user in accordance with one or more embodiments.

FIG. 20-1 illustrates a further example manually-controllable catheter in accordance with one or more embodiments.

FIG. 20-2 illustrates example internal components of the manually-controllable catheter of FIG. 20-1 in accordance with one or more embodiments.

### DETAILED DESCRIPTION

The headings provided herein are for convenience only and do not necessarily affect the scope or meaning of the disclosure. Although certain embodiments and examples are disclosed below, the subject matter extends beyond the specifically disclosed embodiments to other alternative embodiments and/or uses and to modifications and equivalents thereof. Thus, the scope of the claims that may arise here from is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that are similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

The present disclosure relates to aspiration/irrigation catheters/devices. With respect to percutaneous-access devices and other medical devices relevant to the present disclosure, the term “device” is used according to its broad and ordinary meaning and may refer to any type of tool, instrument, assembly, system, apparatus, component, or the like. In some contexts herein, the term “instrument” may be used substantially interchangeably with the term “device.”

Although certain aspects of the present disclosure are described in detail herein in the context of renal, urological, and/or nephrological procedures, such as kidney stone removal/treatment procedures, it should be understood that such context is provided for convenience, and the concepts disclosed herein are applicable to any suitable medical procedures, such as a bronchoscopy. However, as mentioned, description of the renal/urinary anatomy and associated medical issues and procedures is presented below to aid in the description of the concepts disclosed herein.

Kidney stone disease, also known as urolithiasis, is a medical condition that involves the formation in the urinary tract of a solid piece of material, referred to as “kidney stones,” “urinary stones,” “renal calculi,” “renal lithiasis,” or “nephrolithiasis.” Urinary stones may be formed and/or found in the kidneys, the ureters, and the bladder (referred to as “bladder stones”). Such urinary stones can form as a result of mineral concentration in urinary fluid and can cause significant abdominal pain once such stones reach a size sufficient to impede urine flow through the ureter or urethra. Urinary stones may be formed from calcium, magnesium, ammonia, uric acid, cystine, and/or other compounds or combinations thereof.

Several methods can be used for treating patients with kidney stones, including observation, medical treatments (such as expulsion therapy), non-invasive treatments (such as extracorporeal shock wave lithotripsy (ESWL)), minimally-invasive or surgical treatments (such as ureteroscopy and percutaneous nephrolithotomy (“PCNL”)), and so on. In some approaches (e.g., ureteroscopy and PCNL), the physician gains access to the stone, the stone is broken into smaller pieces or fragments, and the relatively small stone fragments/particulates are extracted from the kidney using a basketing device and/or aspiration.

In ureteroscopy procedures, a physician may insert a ureteroscope into the urinary tract through the urethra to remove urinary stones from the bladder and ureter. Typically, a ureteroscope includes an imaging device at its distal end configured to enable visualization of the urinary tract. The ureteroscope can also include a lithotripsy device to capture or break apart urinary stones. During a ureteroscopy procedure, one physician/technician may control the position of the ureteroscope, while another other physician/technician may control the lithotripsy device(s).

In PCNL procedures, which may be used to remove relatively large stones, a physician may insert a nephroscope through the skin (i.e., percutaneously) and intervening tissue to provide access to the treatment site for breaking-up and/or removing the stone(s). During PCNL procedures, fluidics can be applied to clear stone dust, small fragments, and/or thrombus from the treatment site and/or the visual field. In some instances, a relatively straight and/or rigid nephroscope is used, wherein the physician positions the tip of the nephroscope at the appropriate location within the kidney (e.g., calyx) by pushing/leveraging the device against the patient's body. This movement can be harmful to the patient (e.g., cause tissue damage).

In other procedures, such as one or more of those discussed in further detail below, a physician can use multiple instruments via a percutaneous and/or direct access path to remove a kidney stone. For example, a physician can navigate a scope to a target site in a kidney through the urethra in a patient and insert a catheter device into the target site through the skin of the patient. The physician can use the scope and the catheter device in cooperation to fragment the kidney stone and extract the fragments from the patient.

The present disclosure relates to systems, devices, and methods for navigating to and/or aspirating/irrigating a target site to perform a medical procedure. For example, a catheter can be implemented that includes an elongate shaft and a handle/base coupled to the shaft and configured to control actuation of the shaft (at least at a distal portion of the shaft). The shaft can include a lumen configured to couple to an aspiration/irrigation system to provide aspiration/irrigation to a target site, such as to remove an object from a patient. The handle/base of the catheter can be controlled robotically and/or manually to articulate the distal portion of the shaft, so that the catheter can be navigated within the anatomy of a patient. For instance, the catheter can include multiple pull wires or other elongate movement members that are coupled to the distal portion of the shaft and one or more manipulation components in the handle of the catheter. The pull wires/elongate movement members can be manipulated (using the handle) to control movement of the distal portion of the shaft. Additionally, or alternatively, the handle of the catheter can be moved to control movement of the distal portion of the catheter, such as to insert/retract the tip of the catheter.

In some embodiments, the techniques and devices discussed herein can enable objects to be removed from patients in an efficient manner that prevents damage to the anatomy of the patients and/or damage to the removal devices. For example, the articulable catheter structures discussed herein can enable a physician to navigate a distal portion of a catheter within a patient without moving an entirety of the catheter (e.g., by controlling one or more elements within a handle/base of the catheter). In contrast, some nephoscopy procedures require a physician to leverage a proximal portion of a nephroscope to place a tip of the nephroscope in the appropriate location within the patient, resulting in damage to the anatomy of the patient.

In some implementations, the techniques discussed herein implement robotic-assisted medical procedures, wherein robotic tools enable a physician to perform endoscopic and/or percutaneous access and/or treatment for a target anatomical site. For example, the robotic tools can engage with and/or control one or more medical instruments, such as a scope, catheter, or another instrument, to access a target site in a patient and/or perform a treatment at the target site. In some cases, the robotic tools are guided/controlled by a physician. In other cases, the robotic tools operate in an automatic or semi-automatic manner. Although some techniques are discussed in the context of robotic-assisted medical procedures, the techniques may be applicable to other types of medical procedures, such as procedures that do not implement robotic tools or implement robotic tools for relatively few operations (e.g., less than a threshold number). For example, the techniques can be applicable to procedures in which a manually operated medical instrument is implemented, such as a manual catheter and/or scope controlled entirely by a physician.

Certain aspects of the present disclosure are described herein in the context of renal, urological, and/or nephrological procedures, such as kidney stone removal/treatment procedures. However, it should be understood that such context is provided for convenience, and the concepts disclosed herein are applicable to any suitable medical procedure. For example, the following description is also applicable to other surgical/medical operations or medical procedures concerned with the removal of objects from a patient, including any object that can be removed from a treatment site or patient cavity (e.g., the esophagus, ureter, intestine, eye, etc.) via percutaneous and/or endoscopic access, such as, for example, gallbladder stone removal, lung (pulmonary/transthoracic) tumor biopsy, cataract removal, etc. However, as mentioned, description of the renal/urinary anatomy and associated medical issues and procedures is presented below to aid in the description of the concepts disclosed herein.

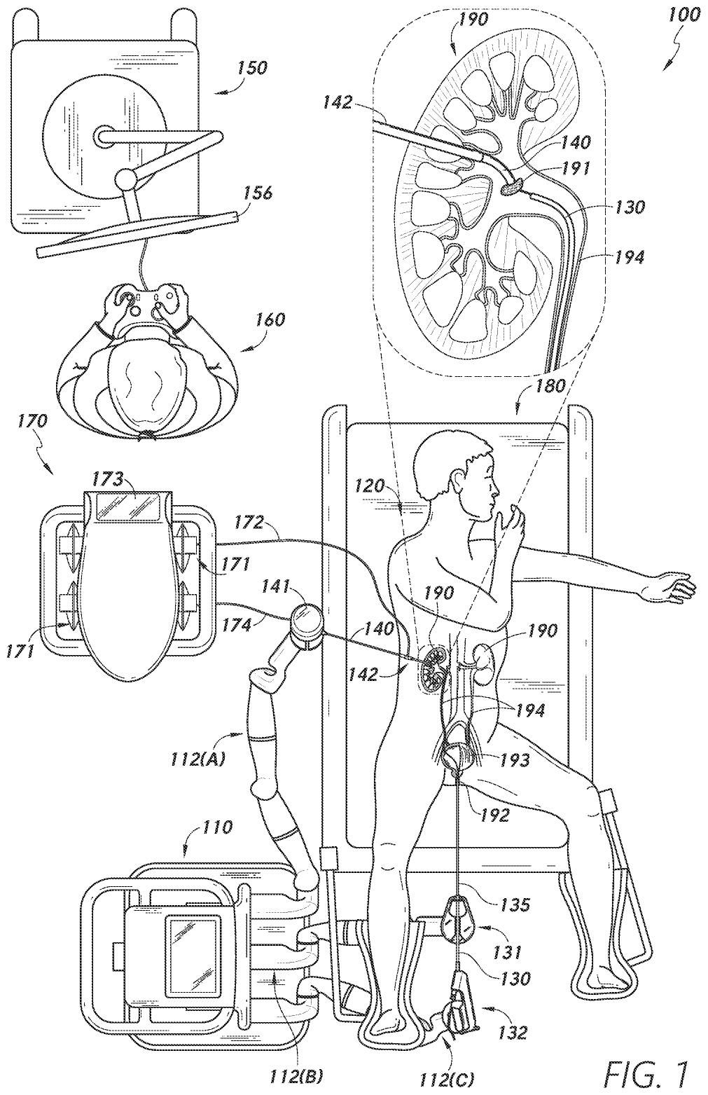

FIG. 1 illustrates an example robotic medical system 100 arranged for a diagnostic and/or therapeutic ureteroscopy procedure in accordance with one or more embodiments. The medical system 100 includes a robotic system 110 configured to engage with and/or control one or more medical instruments/devices to perform a procedure on a patient 120. In the example of FIG. 1, the robotic system 110 couples to a scope 130 and a catheter 140. However, the robotic system 110 can couple to any type of medical instrument. The medical system 100 also includes a control system 150 configured to interface with the robotic system 110 and/or a physician 160, provide information regarding the procedure, and/or perform a variety of other operations. For example, the control system 150 can include a display(s) 156 configured to present certain information to assist the physician 160 in performing the procedure. The medical system 100 can also include a fluid management system 170 (sometimes referred to as “the aspiration system 170” or “the irrigation system 170”) configured to provide aspiration and/or irrigation to a target site, such as via the catheter 140, the scope 130, an instrument/device 142, and/or another instrument/device. The medical system 100 can include a table 180 (e.g., bed) to hold the patient 120. Various acts are described herein as being performed by the physician 160. These acts can be performed directly by the physician 160, a user under the direction of the physician 160, another user (e.g., a technician), a combination thereof, and/or any other user. The devices/components of the medical system 100 can be arranged in a variety of ways depending on the type procedure, phase of the procedure, user preferences, and so on.

The control system 150 can generally operate in cooperation with the robotic system 110 to perform the medical procedure. For example, the control system 150 can communicate with the robotic system 110 via a wireless or wired connection to control a medical instrument connected to the robotic system 110, receive an image(s) captured by a medical instrument, and so on. For example, the control system 150 can receive image data from the scope 130 (e.g., an imaging device associated with the scope 130) and display the image data (and/or representations generated therefrom) to the physician 160 to assist the physician 160 in navigating the scope 130 and/or the catheter 140 within the patient 120. The physician 160 can provide input via an input/output (I/O) device, such as a controller, and the control system 150 can send control signals to the robotic system 110 to control movement of the scope 130/catheter 140 connected to the robotic system 110. The scope 130/catheter 140 (and/or another medical instrument) can be configured to move in a variety of manners, such as to articulate, roll, and so on.

In some embodiments, the control system 150 can provide power to the robotic system 110 via one or more electrical connections, provide optics to the robotic system 110 via one or more optical fibers or other components, and so on. In examples, the control system 150 can communicate with a medical instrument to receive sensor data (via the robotic system 110 and/or directly from the medical instrument). Sensor data can indicate or be used to determine a position and/or orientation of the medical instrument. Further, in examples, the control system 150 can communicate with the table 180 to position the table 180 in a particular orientation or otherwise control the table 180. Moreover, in examples, the control system 150 can communicate with an EM field generator (not illustrated) to control generation of an EM field around the patient 120.

The robotic system 110 can include one or more robotic arms 112 configured to engage with and/or control a medical instrument(s)/device. Each robotic arm 112 can include multiple arm segments coupled to joints, which can provide multiple degrees of movement. A distal end of a robotic arm 112 (e.g., end effector) can be configured to couple to an instrument/device. In the example of FIG. 1, the robotic arm 112(A) is coupled to a handle 141 of the catheter 140. The second robotic arm 112(B) is coupled to a scope-driver instrument coupling/device 131, which can facilitate robotic control/advancement of the scope 130. Further, the third robotic arm 112(C) is coupled to a handle 132 of the scope 130, which can be configured to facilitate advancement and/or operation of the scope 130 and/or a medical instrument that can be deployed through the scope 130, such as an instrument deployed through a working channel of the scope 130. In this example, the second robotic arm 112(B) and/or the third robotic arm 112(C) can control movement of the scope 130 (e.g., articulation, roll, etc.). Although three robotic arms are connected to particular medical instruments in FIG. 1, the robotic system 110 can include any number of robotic arms that are configured to connect to any type of medical instrument/device.

The robotic system 110 can be communicatively coupled to any component of the medical system 100. For example, the robotic system 110 can be communicatively coupled to the control system 150 to receive a control signal from the control system 150 to perform an operation, such as to control a robotic arm 112 in a particular manner, manipulate a medical instrument, and so on. Further, the robotic system 110 can be configured to receive an image (also referred to as image data) from the scope 130 depicting internal anatomy of the patient 120 and/or send the image to the control system 150, which can then be displayed on the display(s) 156. Moreover, the robotic system 110 can be coupled to a component of the medical system 100, such as the control system 150 and/or the fluid management system 170, in a manner as to allow for fluids, optics, power, data, or the like to be received therefrom.

The fluid management system 170 can be configured to provide/control aspiration and/or irrigation to a target site. As shown, the fluid management system 170 can be configured to hold one or more fluid bags/containers 171 and/or control fluid flow thereto/therefrom. For example, an irrigation line 172 may be coupled to one or more of the bags/containers 171 and to an irrigation port of a percutaneous-access device/assembly 142. Irrigation fluid may be provided to the target anatomy via the irrigation line 172 and the percutaneous-access device/assembly 142. The fluid management system 170 may include certain electronic components, such as a display 173, flow control mechanics, and/or certain associated control circuitry. The fluid management cart 170 may comprise a stand-alone tower/cart and may have one or more IV bags 171 hanging on one or more sides thereof. The cart 170 may include a pump with which aspiration fluid may be pulled into a collection container/cartridge via an aspiration channel/tube 174. The aspiration channel/tube 174 may be coupled to the catheter handle 141 to facilitate aspiration via a lumen in the catheter 140.

In the illustrated system 100, the percutaneous-access device 142 is implemented to provide percutaneous access to a kidney 190 of the patient 120. The percutaneous-access instrument 142 may include one or more sheaths and/or shafts through which instruments and/or fluids may access the target anatomy in which the distal end of the instrument 142 is disposed. In this example, the catheter 140 accesses the renal anatomy through the percutaneous-access device 142. That is, the catheter 140 is inserted into the instrument 142 to access the target site.

Although various examples are discussed in the context of providing irrigation/aspiration via the catheter 140 and/or the percutaneous-access device/assembly 142, irrigation fluid and/or aspiration may be provided to the treatment site (e.g., kidney) through another device, such as the scope 130, in some cases. Furthermore, irrigation and aspiration may or may not be provided through the same instrument(s). Where one or more of instruments provides the irrigation and/or aspiration functionality, one or more others of the instruments may be used for other functionality, such as breaking-up the object to be removed.

A medical instrument can include a variety of types of instruments, such as a scope (sometimes referred to as an “endoscope”), a catheter, a needle, a guidewire, a lithotripter, a basket retrieval device, forceps, a vacuum, a needle, a scalpel, an imaging probe, an imaging device, jaws, scissors, graspers, needle holder, micro dissector, staple applier, tacker, suction/irrigation tool, clip applier, and so on. A medical instrument can include a direct entry instrument, percutaneous entry instrument, and/or another type of instrument. In some embodiments, a medical instrument is a steerable device, while in other embodiments a medical instrument is a non-steerable device. In some embodiments, a surgical tool refers to a device that is configured to puncture or to be inserted through the human anatomy, such as a needle, a scalpel, a guidewire, and so on. However, a surgical tool can refer to other types of medical instruments.

The term “scope” or “endoscope” can refer to any type of elongate medical instrument having image generating, viewing, and/or capturing functionality (or configured to provide such functionality with an imaging device deployed though a working channel) and configured to be introduced into any type of organ, cavity, lumen, chamber, and/or space of a body. For example, a scope or endoscope, such as the scope 130, can refer to a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), a borescope, and so on. A scope/endoscope, in some instances, may comprise a rigid or flexible tube and/or may be dimensioned to be passed within an outer sheath, catheter, introducer, or other lumen-type device, or may be used without such devices. In some embodiments, a scope includes one or more working channels through which additional tools/medical instruments, such as lithotripters, basketing devices, forceps, laser devices, imaging devices, etc., can be introduced into a treatment site.

The terms “direct entry” or “direct access” can refer to any entry of instrumentation through a natural or artificial opening in a patient's body. For example, the scope 130 may be referred to as a direct access instrument, since the scope 130 enters into the urinary tract of a patient via the urethra.

The terms “percutaneous entry” or “percutaneous access” can refer to entry, such as by puncture and/or minor incision, of instrumentation through the skin of a patient and any other body layers necessary to reach a target anatomical location associated with a procedure (e.g., the calyx network of the kidney). As such, a percutaneous access instrument may refer to a medical instrument, device, or assembly that is configured to puncture or to be inserted through skin and/or other tissue/anatomy, such as a needle, scalpel, guidewire, sheath, shaft, scope, catheter, and the like. However, it should be understood that a percutaneous access instrument can refer to other types of medical instruments in the context of the present disclosure. In some embodiments, a percutaneous access instrument refers to an instrument/device that is inserted or implemented with a device that facilitates a puncture and/or minor incision through the skin of a patient. For example, the catheter 140 may be referred to as a percutaneous access instrument when the catheter 140 is inserted through a sheath/shaft that is inserted into the skin of a patient.

In some embodiments, a medical instrument includes a sensor (also referred to as a “position sensor”) that is configured to generate sensor data. In examples, sensor data can indicate a position and/or orientation of the medical instrument and/or can be used to determine a position and/or orientation of the medical instrument. For instance, sensor data can indicate a position and/or orientation of a scope, which can indicate a roll of a distal end of the scope. A position and orientation of a medical instrument can be referred to as a pose of the medical instrument. A sensor can be positioned on a distal end of a medical instrument and/or any other location. In some embodiments, a sensor can provide sensor data to the control system 150, the robotic system 110, and/or another system/device to perform one or more localization techniques to determine/track a position and/or an orientation of a medical instrument.

In some embodiments, a sensor can include an electromagnetic (EM) sensor with a coil of conductive material. Here, an EM field generator can provide an EM field that is detected by the EM sensor on the medical instrument. The magnetic field can induce small currents in coils of the EM sensor, which can be analyzed to determine a distance and/or angle/orientation between the EM sensor and the EM field generator. Further, a sensor can include another type of sensor, such as a camera, a range sensor (e.g., depth sensor), a radar device, a shape sensing fiber, an accelerometer, a gyroscope, an accelerometer, a satellite-based positioning sensor (e.g., a global positioning system (GPS)), a radio-frequency transceiver, and so on.

In some embodiments, the medical system 100 can also include an imaging device (not illustrated in FIG. 1) which can be integrated into a C-arm and/or configured to provide imaging during a procedure, such as for a fluoroscopy-type procedure. The imaging device can be configured to capture/generate one or more images of the patient 120 during a procedure, such as one or more x-ray or CT images. In examples, images from the imaging device can be provided in real-time to view anatomy and/or medical instruments within the patient 120 to assist the physician 160 in performing a procedure. The imaging device can be used to perform a fluoroscopy (e.g., with a contrast dye within the patient 120) or another type of imaging technique.

The various components of the medical system 100 can be communicatively coupled to each other over a network, which can include a wireless and/or wired network. Example networks include one or more personal area networks (PANs), local area networks (LANs), wide area networks (WANs), Internet area networks (LANs), body area networks (BANs), cellular networks, the Internet, etc. Further, in some embodiments, the components of the medical system 100 are connected for data communication, fluid/gas exchange, power exchange, and so on, via one or more support cables, tubes, or the like.

In some examples, the medical system 100 is implemented to perform a medical procedure relating to the renal anatomy, such as to treat kidney stones. For instance, robotic-assisted percutaneous procedures can be implemented, wherein robotic tools (e.g., one or more components of the medical system 100) can enable a physician/urologist to perform endoscopic (e.g., ureteroscopy) target access as well as percutaneous access/treatment. This disclosure, however, is not limited to kidney stone removal and/or robotic-assisted procedures. In some implementations, robotic medical solutions can provide relatively higher precision, superior control, and/or superior hand-eye coordination with respect to certain instruments compared to strictly manual procedures. For example, robotic-assisted percutaneous access to the kidney in accordance with some procedures can advantageously enable a urologist to perform both direct-entry endoscopic renal access and percutaneous renal access. Although some embodiments of the present disclosure are presented in the context of catheters, nephroscopes, ureteroscopes, and/or the human renal anatomy, it should be understood that the principles disclosed herein may be implemented in any type of endoscopic/percutaneous procedure or another type of procedure.

In one illustrative and non-limiting procedure, the medical system 100 can be used to remove a kidney stone 191 from the patient 120. During setup for the procedure, the physician 160 can position the robotic arms 112 of the robotic system 110 in the desired configuration and/or attach the appropriate medical instruments. For example, the physician 160 can position the first robotic arm 112(A) near a treatment site and attach an EM field generator (not illustrated), which can assist in tracking a location of the scope 130 and/or other instruments/devices during the procedure. Further, the physician 160 can position the second robotic arm 112(B) between the legs of the patient 120 and attach the scope-driver instrument coupling 131, which can facilitate robotic control/advancement of the scope 130. In some instances, the physician 160 can insert a sheath/access instrument 135 into the urethra 192 of the patient 120 and/or through the bladder 193 and up the ureter 194. The physician 160 can connect the sheath/access instrument 135 to the scope-drive instrument coupling 131. The sheath/access instrument 135 can include a lumen-type device configured to receive the scope 130, thereby assisting in inserting the scope 130 into the anatomy of the patient 120. However, in some embodiments the sheath/access instrument 135 is not used (e.g., the scope 130 is inserted directly into the urethra 192). The physician 160 can then insert the scope 130 into the sheath/access 135 instrument manually, robotically, or a combination thereof. The physician 160 can attach the handle 132 of the scope 130 to the third robotic arm 112(C), which can be configured to facilitate advancement and/or operation of a basketing device, laser device, and/or another medical instrument deployed through the scope 130.

The physician 160 can interact with the control system 150 to cause the robotic system 110 to advance and/or navigate the scope 130 into the kidney 190. For example, the physician 160 can navigate the scope 130 using a controller or other I/O device to locate the kidney stone 191. The control system 150 can provide information via the display(s) 156 regarding the scope 130 to assist the physician 160 in navigating the scope 130, such as to view an image representation (e.g., a real-time image(s) captured by the scope 130). In some embodiments, the control system 150 can use localization techniques to determine a position and/or an orientation of the scope 130, which can be viewed by the physician 160 through the display(s) 156, in some cases. Further, other types of information can also be presented through the display(s) 156 to assist the physician 160 in controlling the scope 130, such as x-ray images of the internal anatomy of the patient 120.

Once at the site of the kidney stone 191 (e.g., within the calyx of the kidney 190), the scope 130 can be used to designate/tag a target location for a catheter to access the kidney 190 percutaneously. To minimize damage to the kidney 190 and/or the surrounding anatomy, the physician 160 can designate a papilla as the target location for entering into the kidney 190 percutaneously. However, other target locations can be designated or determined. In some embodiments of designating the papilla, the physician 160 can navigate the scope 130 to contact the papilla, the control system 150 can use localization techniques to determine a location of the scope 130 (e.g., a location of the distal end of the scope 130), and the control system 150 can associate the location of the scope 130 with the target location. Further, in some embodiments, the physician 160 can navigate the scope 130 to be within a particular distance to the papilla (e.g., park in front of the papilla) and provide input indicating that the target location is within a field-of-view of the scope 130. The control system 150 can perform image analysis and/or other localization techniques to determine a location of the target location. Moreover, in some embodiments, the scope 130 can deliver a fiduciary to mark the papilla as the target location.

When the target location is designated, the catheter 140 can be inserted through a percutaneous access path into the patient 120 to reach the target site (e.g., rendezvous with the scope 130). For example, the catheter 140 can be connected to the first robotic arm 112(A) (upon removing the EM field generator) and the physician 160 can interact with the control system 150 to cause the robotic system 110 to advance and/or navigate the catheter 140, as shown in FIG. 1. Alternatively, or additionally, the catheter 140 can be manually inserted and/or controlled, such as when the catheter 140 is implemented as a manually-controllable catheter. In some embodiments, a needle or another medical instrument is inserted into the patient 120 to create the percutaneous access path. The control system 150 can provide information via the display(s) 156 regarding the catheter 140 to assist the physician 160 in navigating the catheter. For example, the display(s) 156 can provide image data from the perspective of the scope 130, wherein the image data may depict the catheter 140 (e.g., when within the field-of-view of an imaging device of the scope 130).

With the scope 130 and/or the catheter 140 located at the target location, the physician 160 can use the scope 130 to break up the kidney stone 191 and/or use the catheter 140 to extract pieces of the kidney stone 191 from the patient 120. For example, the scope 130 can deploy a tool (e.g., a laser, a cutting instrument, lithotripter, etc.) through a working channel to fragment the kidney stone 191 into pieces and the catheter 140 can suck out the pieces from the kidney 190 through the percutaneous access path. The catheter 140 can provide aspiration to maintain/hold the kidney stone 191 at a distal end of the catheter 140 and/or at a relatively fixed position, while the scope 130 fragments the kidney stone 191 using a tool (e.g., laser), as shown in FIG. 1. The fluid management system 170 can provide irrigation to the target site via the percutaneous-access device/assembly 142 and/or provide aspiration to the target site via the catheter 140 (e.g., a lumen in the catheter 140).

Although various example procedures are discussed in the context of implementing a robotically controlled catheter 140, the procedure can be implemented with a manually controllable catheter. For example, the catheter 140 can include a manually controllable handle that is configured to be held/manipulated by the physician 160. The physician 160 can navigate the catheter 140 by rolling, inserting, retracting, or otherwise manipulating the handle and/or a manual actuator, which can result in articulation of a distal portion of the catheter 140. Example robotically controllable and manually controllable catheters are discussed in further detail below.

The medical system 100 (and/or other medical systems discussed herein) can provide a variety of benefits, such as providing guidance to assist a physician in performing a procedure (e.g., instrument tracking, instrument navigation, instrument calibration, etc.), enabling a physician to perform a procedure from an ergonomic position without the need for awkward arm motions and/or positions, enabling a single physician to perform a procedure with one or more medical instruments, avoiding radiation exposure (e.g., associated with fluoroscopy techniques), enabling a procedure to be performed in a single-operative setting, providing continuous aspiration/irrigation to remove an object more efficiently (e.g., to remove a kidney stone), and so on. For example, the medical system 100 can provide guidance information to assist a physician in using various medical instruments to access a target anatomical feature while minimizing bleeding and/or damage to anatomy (e.g., critical organs, blood vessels, etc.). Further, the medical system 100 can provide non-radiation-based navigational and/or localization techniques to reduce physician and patient exposure to radiation and/or reduce the amount of equipment in the operating room. Moreover, the medical system 100 can provide functionality that is distributed between at least the control system 150 and the robotic system 110, which can be independently movable. Such distribution of functionality and/or mobility can enable the control system 150 and/or the robotic system 110 to be placed at locations that are optimal for a particular medical procedure, which can maximize working area around the patient and/or provide an optimized location for a physician to perform a procedure.

Although various techniques/systems are discussed as being implemented as robotically-assisted procedures (e.g., procedures that at least partly use the medical system 100), the techniques/systems can be implemented in other procedures, such as in fully-robotic medical procedures, human-only procedures (e.g., free of robotic systems), and so on. For example, the medical system 100 can be used to perform a procedure without a physician holding/manipulating a medical instrument and without a physician controlling movement of a robotic system/arm (e.g., a fully-robotic procedure that relies on relatively little input to direct the procedure). That is, medical instruments that are used during a procedure can each be held/controlled by components of the medical system 100, such as the robotic arms 112 of the robotic system 110.

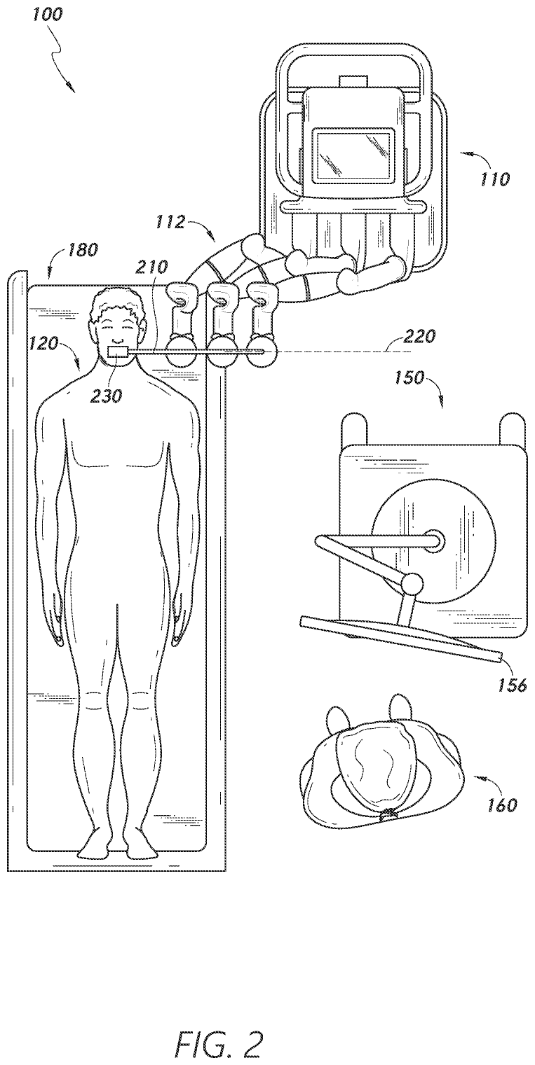

FIG. 2 illustrates the example robotic medical system 100 arranged for a diagnostic and/or therapeutic bronchoscopy procedure in accordance with one or more embodiments. During a bronchoscopy, the arm(s) 112 of the robotic system 110 may be configured to deliver a medical instrument, such as a steerable endoscope 210, which may be a procedure-specific bronchoscope for bronchoscopy, to a natural orifice access point (i.e., the mouth of the patient 120 positioned on the table 180 in the present example) to deliver diagnostic and/or therapeutic tools. As shown, the robotic system 110 (e.g., cart) may be positioned proximate to the patient's upper torso in order to provide access to the access point. Similarly, the robotic arms 112 may be actuated to position the bronchoscope 210 relative to the access point. The arrangement in FIG. 2 may also be utilized when performing a gastro-intestinal (GI) procedure with a gastroscope, a specialized endoscope for GI procedures.

Once the robotic system 110 is properly positioned, the robotic arms 112 may insert the steerable endoscope 210 into the patient robotically, manually, or a combination thereof. The steerable endoscope 210 may comprise at least two telescoping parts, such as an inner leader portion and an outer sheath portion, with each portion coupled to a separate instrument driver from a set of instrument drivers and/or with each instrument driver coupled to the distal end of a respective robotic arm 112. This linear arrangement of the instrument drivers creates a “virtual rail” 220 that may be repositioned in space by manipulating the one or more robotic arms 112 into different angles and/or positions. The virtual rails/paths described herein are depicted in the figures using dashed lines that generally do not depict any physical structure of the system. Translation of one or more of the instrument drivers along the virtual rail 220 can advance or retract the endoscope 210 from the patient 120.

The endoscope 210 may be directed down the patient's trachea and lungs after insertion using precise commands from the robotic system 110 until reaching the target operative site. The use of separate instrument drivers can allow independent driving of separate portions of the endoscope/assembly 210. For example, the endoscope 210 may be directed to deliver a biopsy needle to a target, such as, for example, a lesion or nodule within the lungs of a patient. The needle may be deployed down a working channel that runs the length of the endoscope 210 to obtain a tissue sample to be analyzed by a pathologist. Depending on the pathology results, additional tools may be deployed down the working channel of the endoscope 210 for additional biopsies. For example, when a nodule is identified as being malignant, the endoscope 210 may endoscopically deliver tools to resect the potentially cancerous tissue. In some instances, diagnostic and therapeutic treatments can be delivered in separate procedures. In those circumstances, the endoscope 210 may also be used to deliver a fiducial to “mark” the location of the target nodule as well. In other instances, diagnostic and therapeutic treatments may be delivered during the same procedure.

In the arrangement of the system 100 in FIG. 2, a patient introducer 230 is attached to the patient 120 via a port (not shown; e.g., surgical tube). The patient introducer 230 may be secured to the table 180 (e.g., via a patient introducer holder configured to support the introducer 230 and secure the position of the patient introducer 230 with respect to the table 180 or other structure). In some embodiments, the patient introducer 230 may include a proximal end, a distal end, and an introducer tube therebetween. The proximal end of the patient introducer 230 can provide an opening/orifice which may be configured to receive the instrument 210 (e.g., bronchoscope), and the distal end of the patient introducer 230 can provide a second opening which may be configured to guide the instrument 210 into the patient-access port. A curved tube component of the introducer 230 can connect the proximal and distal ends thereof and guide the instrument 210 through the introducer 230.

The curvature of the introducer 230 may enable the robotic system 110 to manipulate the instrument 210 from a position that is not in direct axial alignment with the patient-access port, thereby allowing for greater flexibility in the placement of the robotic system 110 within the room. Further, the curvature of the introducer 230 may allow the robotic arms 112 of the robotic system 110 to be substantially horizontally aligned with the patient introducer 230, which may facilitate manual movement of the robotic arm(s) 112 if needed.

In some embodiments, one or more of the catheters discussed herein can be implemented in a bronchoscopy procedure, such as that illustrated in FIG. 2. For example, a catheter can be implemented in cooperation with or instead of the endoscope 210 to remove an object from the patient 120. In one illustration, a catheter and the endoscope 210 are interchanged on the robotic arms 112 and separately used to investigate/treat a target site. Here, the catheter can be inserted through the patient introducer 230 and used to provide aspiration/irrigation, such as to remove an object from the patient 120. In another illustration, a catheter is deployed through a working channel on the endoscope 210 to provide irrigation/aspiration.

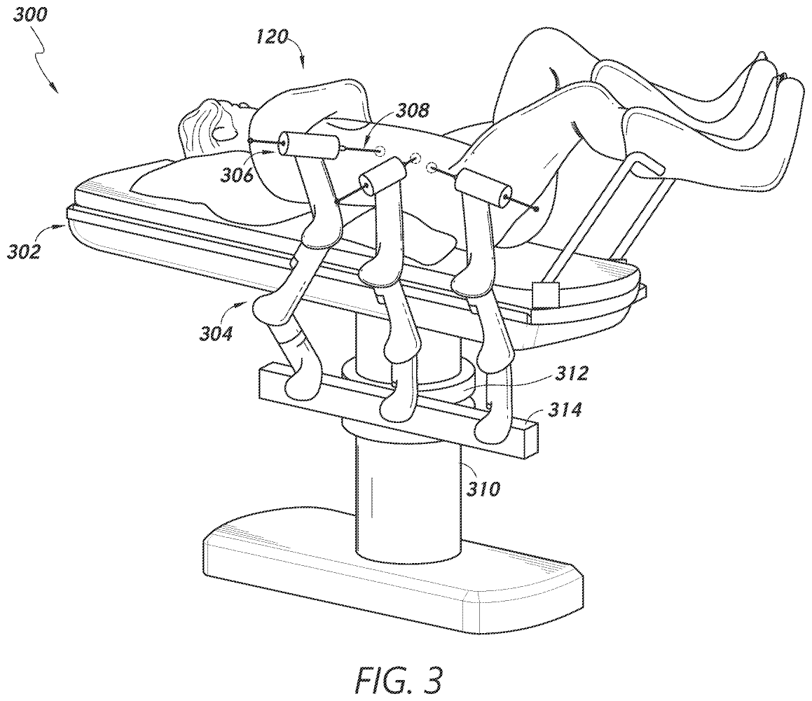

FIG. 3 illustrates a table-based robotic system 300 configured to perform a medical procedure in accordance with one or more embodiments. Here, one or more of the robotic components of the robotic medical system 100 can be incorporated into a table 302, which can reduce the amount of capital equipment within an operating room and/or allow greater access to the patient 120, in comparison to cart-based robotic systems. For example, the system 300 can include one or more components of the control system 150, the robotic system 110, and/or the fluid management system 170.

As shown, the table 302 can include/incorporate one or more robotic arms 304 configured to engage with and/or control a medical instrument(s)/device. Each robotic arm 304 can include multiple arm segments coupled to joints, which can provide multiple degrees of movement. A distal end of a robotic arm 304 (i.e., end effector 306) can be configured to couple to an instrument/device, which can include any of the medical instruments/devices discussed herein, such as a catheter, needle, scope, etc. Each robotic arm 304 can be similar to or different than the robotic arms 112 of the system 100 of FIGS. 1 and 2. Further, each end effector 306 can be similar to or different than an end effector of the robotic system 100.

As shown, the robotic-enabled table system 300 can include a column 310 coupled to one or more carriages 312 (e.g., ring-shaped movable structures), from which the one or more robotic arms 304 may emanate. The carriage(s) 312 may translate along a vertical column interface that runs at least a portion of the length of the column 310 to provide different vantage points from which the robotic arms 304 may be positioned to reach the patient 120. The carriage(s) 312 may rotate around the column 310 in some embodiments using a mechanical motor positioned within the column 310 to allow the robotic arms 304 to have access to multiples sides of the table 302. Rotation and/or translation of the carriage(s) 312 can allow the system 300 to align the medical instruments, such as endoscopes and/or catheters, into different access points on the patient 120. By providing vertical adjustment, the robotic arms 304 can be configured to be stowed compactly beneath the platform of the table system 300 and subsequently raised during a procedure. The robotic arms 304 may be mounted on the carriage(s) 312 through one or more arm mounts 314, which may comprise a series of joints that may individually rotate and/or telescopically extend to provide additional configurability to the robotic arms 304. The column 310 structurally provides support for the table platform and a path for vertical translation of the carriage(s) 312. The column 310 may also convey power and control signals to the carriage(s) 312 and/or the robotic arms 304 mounted thereon.

In some embodiments, the table-based robotic system 300 can include or be associated with a control system, similar to the control system 150, to interface with a physician and/or provide information regarding a medical procedure. For example, a control system can include an input component(s) to enable a physician to control the one or more robotic arms 304 and/or medical instruments attached to the one or more robotic arms 304. In some implementations, the input component(s) enables the physician to provide input to control a medical instrument in a similar manner as if the physician were physically holding/manipulating the medical instrument.

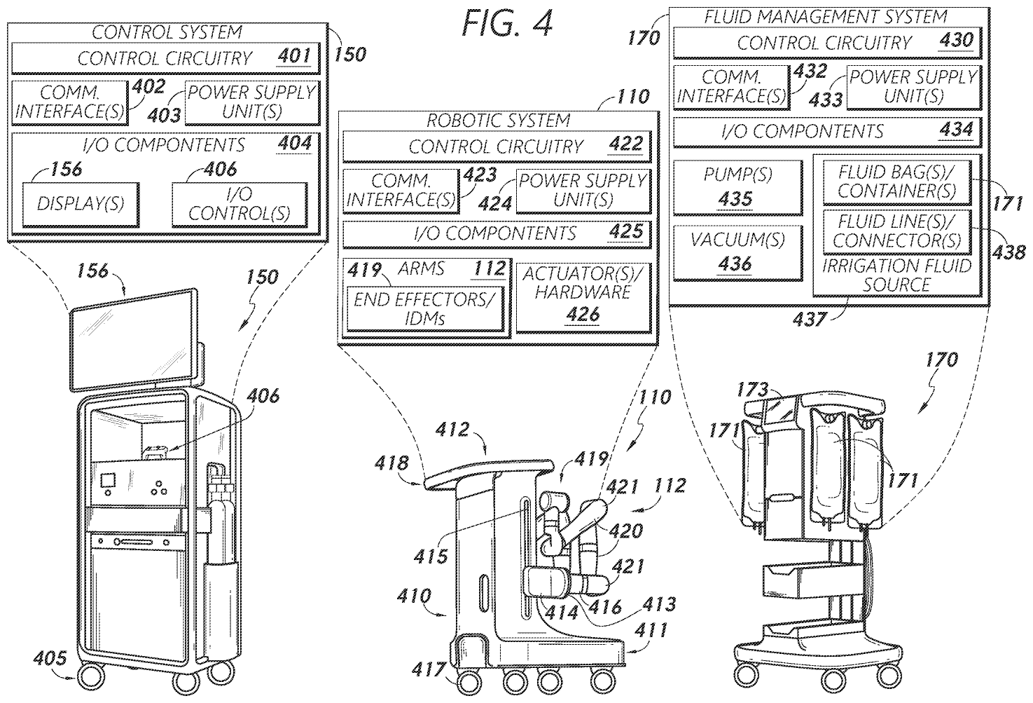

FIG. 4 illustrates medical system components that may be implemented in any of the medical systems of FIGS. 1-3 in accordance with one or more embodiments of the present disclosure. Although certain components in FIG. 4, it should be understood that additional components not shown can be included in embodiments in accordance with the present disclosure. Furthermore, any of the illustrated components can be omitted, interchanged, and/or integrated into other devices/systems, such as the table 180, a medical instrument, etc.

The control system 150 can include one or more of the following components, devices, modules, and/or units (referred to herein as “components”), either separately/individually and/or in combination/collectively: control circuitry 401, one or more communication interfaces 402, one or more power supply units 403, one or more I/O components 404, and/or one or more mobilization components 405 (e.g., casters or other types of wheels). In some embodiments, the control system 150 can comprise a housing/enclosure configured and/or dimensioned to house or contain at least part of one or more of the components of the control system 150. In this example, the control system 150 is illustrated as a cart-based system that is movable with the one or more mobilization components 405. In some cases, after reaching the appropriate position, the one or more mobilization components 405 can be immobilized using wheel locks to hold the control system 150 in place. However, the control system 150 can be implemented as a stationary system, integrated into another system/device, and so on.

The various components of the control system 150 can be electrically and/or communicatively coupled using certain connectivity circuitry/devices/features, which may or may not be part of control circuitry. For example, the connectivity feature(s) can include one or more printed circuit boards configured to facilitate mounting and/or interconnectivity of at least some of the various components/circuitry of the control system 150. In some embodiments, two or more of the components of the control system 150 can be electrically and/or communicatively coupled to each other.

The one or more communication interfaces 402 can be configured to communicate with one or more devices/sensors/systems. For example, the one or more communication interfaces 402 can send/receive data in a wireless and/or wired manner over a network. In some embodiments, the one or more communication interfaces 402 can implement a wireless technology, such as Bluetooth, Wi-Fi, near field communication (NFC), or the like.

The one or more power supply units 403 can be configured to manage and/or provide power for the control system 150 (and/or the robotic system 110/fluid management system 170, in some cases). In some embodiments, the one or more power supply units 403 include one or more batteries, such as a lithium-based battery, a lead-acid battery, an alkaline battery, and/or another type of battery. That is, the one or more power supply units 403 can comprise one or more devices and/or circuitry configured to provide a source of power and/or provide power management functionality. Moreover, in some embodiments the one or more power supply units 403 include a mains power connector that is configured to couple to an alternating current (AC) or direct current (DC) mains power source.

The one or more I/O components/devices 404 can include a variety of components to receive input and/or provide output, such as to interface with a user to assist in performing a medical procedure. The one or more I/O components 404 can be configured to receive touch, speech, gesture, or any other type of input. In examples, the one or more I/O components 404 can be used to provide input regarding control of a device/system, such as to control the robotic system 110, navigate a scope/catheter or other medical instrument attached to the robotic system 110 and/or deployed through the scope, control the table 180, control a fluoroscopy device, and so on. For example, a physician (not illustrated) can provide input via the I/O component(s) 404 and, in response, the control system 150 can send control signals to the robotic system 110 to manipulate a medical instrument. In examples, the physician can use the same I/O device to control multiple medical instruments (e.g., switch control between the instruments).

As shown, the one or more I/O components 404 can include the one or more displays 156 (sometimes referred to as “the one or more display devices 156”) configured to display data. The one or more displays 156 can include one or more liquid-crystal displays (LCD), light-emitting diode (LED) displays, organic LED displays, plasma displays, electronic paper displays, and/or any other type(s) of technology. In some embodiments, the one or more displays 156 include one or more touchscreens configured to receive input and/or display data. Further, the one or more I/O components 404 can include one or more I/O devices/controls 406, which can include a touch pad, controller (e.g., hand-held controller, video-game-type controller, finger-based controls that enable finger-like movement, etc.), mouse, keyboard, wearable device (e.g., optical head-mounted display), virtual or augmented reality device (e.g., head-mounted display), foot panel (e.g., buttons at the user's feet), etc. Additionally, the one or more I/O components 404 can include one or more speakers configured to output sounds based on audio signals and/or one or more microphones configured to receive sounds and generate audio signals. In some embodiments, the one or more I/O components 404 include or are implemented as a console.

In some embodiments, the one or more I/O components 404 can output information related to a procedure. For example, the control system 150 can receive real-time images that are captured by a scope and display the real-time images and/or visual/image representations of the real-time images via the display(s) 156. The display(s) 156 can present an interface(s), which can include image data from the scope and/or another medical instrument. Additionally, or alternatively, the control system 150 can receive signals (e.g., analog, digital, electrical, acoustic/sonic, pneumatic, tactile, hydraulic, etc.) from a medical monitor and/or a sensor associated with a patient, and the display(s) 156 can present information regarding the health or environment of the patient. Such information can include information that is displayed via a medical monitor including, for example, a heart rate (e.g., ECG, HRV, etc.), blood pressure/rate, muscle bio-signals (e.g., EMG), body temperature, blood oxygen saturation (e.g., SpO₂), CO₂, brainwaves (e.g., EEG), environmental and/or local or core body temperature, and so on.

In some embodiments, the control system 150 can be coupled to the robotic system 110, a table 180 or another table, and/or a medical instrument, through one or more cables or connections (not shown). In some implementations, support functionality from the control system 150 can be provided through a single cable, simplifying and de-cluttering an operating room. In other implementations, specific functionality can be coupled in separate cabling and connections. For example, while power can be provided through a single power cable, the support for controls, optics, fluidics, and/or navigation can be provided through a separate cable.

The robotic system 110 generally includes an elongate support structure 410 (also referred to as a “column”), a robotic system base 411, and a console 412 at the top of the column 410. The column 410 can include one or more carriages 413 (also referred to as “the arm support 413”) for supporting the deployment of one or more the robotic arms 112. The carriage 413 can include individually configurable arm mounts that rotate along a perpendicular axis to adjust the base of the robotic arms 112 for positioning relative to a patient. The carriage 413 also includes a carriage interface 414 that allows the carriage 413 to vertically translate along the column 410. The carriage interface 414 can be connected to the column 410 through slots, such as slot 415, that are positioned on opposite sides of the column 410 to guide the vertical translation of the carriage 413. The slot 415 can include a vertical translation interface to position and/or hold the carriage 413 at various vertical heights relative to the base 411. Vertical translation of the carriage 413 allows the robotic system 110 to adjust the reach of the robotic arms 112 to meet a variety of table heights, patient sizes, physician preferences. etc. Similarly, the individually configurable arm mounts on the carriage 413 allow a robotic arm base 416 of the robotic arms 112 to be angled in a variety of configurations. The column 410 can internally comprise mechanisms, such as gears and/or motors, that are designed to use a vertically aligned lead screw to translate the carriage 413 in a mechanized fashion in response to control signals generated in response to user inputs, such as inputs from an I/O device(s).

The base 411 can balance the weight of the column 410, the carriage 413, and/or robotic arms 112 over a surface, such as the floor. Accordingly, the base 411 can house heavier components, such as one or more electronics, motors, power supply, etc., as well as components that enable movement and/or immobilize the robotic system 110. For example, the base 411 can include rollable wheels 417 (also referred to as “the casters 417” or “the mobilization components 417”) that allow for the robotic system 110 to move around the room for a procedure. After reaching an appropriate position, the casters 417 can be immobilized using wheel locks to hold the robotic system 110 in place during the procedure. As shown, the robotic system 110 also includes a handle 418 to assist with maneuvering and/or stabilizing the robotic system 110. In this example, the robotic system 110 is illustrated as a cart-based system that is movable. However, the robotic system 110 can be implemented as a stationary system, integrated into a table, and so on.

The robotic arms 112 can generally comprise robotic the arm bases 416 and end effectors 419, separated by a series of linkages 420 (also referred to as “arm segments 420”) that are connected by a series of joints 421. Each joint 421 can comprise an independent actuator and each actuator can comprise an independently controllable motor. Each independently controllable joint 421 represents an independent degree of freedom available to the robotic arm 112. For example, each of the arms 112 can have seven joints, and thus, provide seven degrees of freedom. However, any number of joints can be implemented with any degrees of freedom. In examples, a multitude of joints can result in a multitude of degrees of freedom, allowing for “redundant” degrees of freedom. Redundant degrees of freedom allow the robotic arms 112 to position their respective end effectors 419 at a specific position, orientation, and/or trajectory in space using different linkage positions and/or joint angles. In some embodiments, the end effectors 419 can be configured to engage with and/or control a medical instrument, a device, an object, and so on. The freedom of movement of the arms 112 can allow the robotic system 110 to position and/or direct a medical instrument from a desired point in space and/or allow a physician to move the arms 112 into a clinically advantageous position away from the patient to create access, while avoiding arm collisions.

The end effector 419 of each of the robotic arms 112 can comprise an instrument device manipulator (IDM). In some embodiments, the IDM can be removed and replaced with a different type of IDM. For example, a first type of IDM can manipulate an endoscope, a second type of IDM can manipulate a catheter, a third type of IDM can hold an EM field generator, and so on. However, the same IDM can be used. In some instances, an IDM can include connectors to transfer pneumatic pressure, electrical power, electrical signals, and/or optical signals to/from the robotic arm 112. The IDMs may be configured to manipulate medical instruments using techniques including, for example, direct drives, harmonic drives, geared drives, belts/pulleys, magnetic drives, and the like. In some embodiments, the IDMs can be attached to respective ones of the robotic arms 112, wherein the robotic arms 112 are configured to insert or retract the respective coupled medical instruments into or out of the treatment site.

In some embodiments, the robotic arms 112 can be configured to control a position, orientation, and/or articulation of a medical instrument (e.g., a sheath and/or a leader of a scope) attached thereto. For example, the robotic arms 112 can be configured/configurable to manipulate a scope/catheter using elongate movement members. The elongate movement members can include one or more pull wires, cables, fibers, and/or flexible shafts. To illustrate, the robotic arms 112 can be configured to actuate multiple pull wires of the scope/catheter to deflect the tip of the scope/catheter. Pull wires can include any suitable or desirable materials, such as metallic and/or non-metallic materials such as stainless steel, Kevlar, tungsten, carbon fiber, and the like. In some embodiments, the scope/catheter is configured to exhibit nonlinear behavior in response to forces applied by the elongate movement members. The nonlinear behavior can be based on stiffness and/or compressibility of the scope/catheter, as well as variability in slack or stiffness between different elongate movement members.

As shown, the console 412 is positioned at the upper end of column 410 of the robotic system 110. The console 412 can include a display(s) to provide a user interface for receiving user input and/or providing output (e.g., a dual-purpose device, such as a touchscreen), such as to provide a physician/user with pre-operative data, intra-operative data, information to configure the robotic system 110, and so on. Potential pre-operative data can include pre-operative plans, navigation and mapping data derived from pre-operative computerized tomography (CT) scans, and/or notes from pre-operative patient interviews. Intra-operative data can include optical information provided from a tool, sensor and/or coordinate information from sensors, as well as vital patient statistics, such as respiration, heart rate, and/or pulse. The console 412 can be positioned and tilted to allow a physician to access the console 412 from the side of the column 410 opposite arm base 416. From this position, the physician may view the console 412, robotic arms 112, and patient while operating the console 412 from behind the robotic system 110.

The robotic system 110 can also include control circuitry 422, one or more communication interfaces 423, one or more power supply units 424, one or more input/output components 425, and one or more actuators/hardware 426. The one or more communication interfaces 423 can be configured to communicate with one or more device/sensors/systems. For example, the one or more communication interfaces 423 can send/receive data in a wireless and/or wired manner over a network.

The one or more power supply units 424 can be configured to manage and/or provide power for the robotic system 110. In some embodiments, the one or more power supply units 424 include one or more batteries, such as a lithium-based battery, a lead-acid battery, an alkaline battery, and/or another type of battery. That is, the one or more power supply units 424 can comprise one or more devices and/or circuitry configured to provide a source of power and/or provide power management functionality. Moreover, in some embodiments the one or more power supply units 424 include a mains power connector that is configured to couple to an alternating current (AC) or direct current (DC) mains power source. Further, in some embodiments, the one or more power supply units 424 include a connector that is configured to couple to the control system 150 to receive power from the control system 150.

The one or more I/O components/devices 425 can be configured to receive input and/or provide output, such as to interface with a user. The one or more I/O components 425 can be configured to receive touch, speech, gesture, or any other type of input. In examples, the one or more I/O components 425 can be used to provide input regarding control of a device/system, such as to control/configure the robotic system 110. The one or more I/O components 425 can include the one or more displays configured to display data. The one or more displays can include one or more liquid-crystal displays (LCD), light-emitting diode (LED) displays, organic LED displays, plasma displays, electronic paper displays, and/or any other type(s) of technology. In some embodiments, the one or more displays include one or more touchscreens configured to receive input and/or display data. Further, the one or more I/O components 425 can include a touch pad, controller, mouse, keyboard, wearable device (e.g., optical head-mounted display), virtual or augmented reality device (e.g., head-mounted display), etc. Additionally, the one or more I/O components 425 can include one or more speakers configured to output sounds based on audio signals and/or one or more microphones configured to receive sounds and generate audio signals. In some embodiments, the one or more I/O components 425 include or are implemented as the console 412. Further, the one or more I/O components 425 can include one or more buttons that can be physically pressed, such as a button on a distal end of a robotic arm 112 (which can enable/disable an admittance control mode of the robotic arm 112 for manual manipulation/movement of the robotic arm 112).

The one or more actuators/hardware 426 can be configured to facilitate movement of the robotic arms 112. Each actuator 426 can comprise a motor, which can be implemented in a joint or elsewhere within a robotic arm 112 to facilitate movement of the joint and/or a connected arm segment/linkage. In some embodiments, a user can manually manipulate a robotic arm 112 without using electronic user controls. For example, during setup in a surgical operating room or at any point during a procedure, a user may select a button on a distal end of a robotic arm 112 to enable an admittance control mode and then manually move the robotic arm 112 to a particular orientation/position.

The various components of the robotic system 110 can be electrically and/or communicatively coupled using certain connectivity circuitry/devices/features, which may or may not be part of the control circuitry 422. For example, the connectivity feature(s) can include one or more printed circuit boards configured to facilitate mounting and/or interconnectivity of at least some of the various components/circuitry of the robotic system 110. In some embodiments, two or more of the components of the robotic system 110 can be electrically and/or communicatively coupled to each other.

The robotic fluid management system 170 can include control circuitry 430, one or more communication interfaces 432, one or more power supply units 433, one or more input/output components 434, one or more pumps 435, one or more vacuums 436, and an irrigation fluid source 437. The one or more communication interfaces 432 can be configured to communicate with one or more device/sensors/systems. For example, the one or more communication interfaces 432 can send/receive data in a wireless and/or wired manner over a network.

The one or more power supply units 433 can be configured to manage and/or provide power for the fluid management system 170. In some embodiments, the one or more power supply units 433 include one or more batteries, such as a lithium-based battery, a lead-acid battery, an alkaline battery, and/or another type of battery. That is, the one or more power supply units 433 can comprise one or more devices and/or circuitry configured to provide a source of power and/or provide power management functionality. Moreover, in some embodiments the one or more power supply units 433 include a mains power connector that is configured to couple to an alternating current (AC) or direct current (DC) mains power source. Further, in some embodiments, the one or more power supply units 433 include a connector that is configured to couple to the control system 150 to receive power from the control system 150.

The one or more I/O components/devices 434 can be configured to receive input and/or provide output, such as to interface with a user. The one or more I/O components 434 can be configured to receive touch, speech, gesture, or any other type of input. The one or more I/O components 434 can include a display, a touch pad, controller, mouse, keyboard, wearable device (e.g., optical head-mounted display), virtual or augmented reality device (e.g., head-mounted display), speaker, microphone, etc. Further, the one or more I/O components 434 can include one or more buttons that can be physically pressed.

The fluid management system 170 can be configured to control the pump(s) 435 and/or the vacuum(s) 436 to provide irrigation/aspiration. For example, a medical instrument may be attached to the pump(s) 435/vacuum 436 to provide irrigation/aspiration to a target site via medical instrument. In examples, the fluid management system 170 can include one or more flow meters, valve controls, and/or other fluid-/flow-control components (e.g., sensor devices, such as pressure sensors) in order to provide controlled irrigation and/or aspiration/suction capabilities for a medical instrument. In some embodiments, the control system 150 and/or the robotic system 110 can generate and provide one or more signals to the fluid management system 170 to control irrigation/aspiration.

The pump(s) 435 can be attached to an irrigation fluid source 437, which can include the fluid bag(s)/container(s) 171 and/or a fluid line(s)/connector(s) 438 to connect to a medical instrument(s). The pump(s) 435 can pump irrigation fluid (e.g., saline solution) through one or more medical instruments and into a treatment site. In some examples, the pump(s) 435 is a peristaltic pump(s). In some embodiments, the pump(s) 435 can be replaced with a vacuum that is configured to apply a vacuum pressure to draw the irrigation fluid from the irrigation fluid source 437 and out through the respective coupled medical instrument. Although FIG. 4 includes the pump(s) 435, in some embodiments, irrigation fluid flow is achieved without the use of pumps, wherein such flow is driven primarily by gravitational force.

The vacuum(s) 436 can be configured to facilitate fluid aspiration. For example, the vacuum(s) 436 can be configured to apply a negative pressure to draw fluid out of a treatment site. The vacuum(s) 436 may be connected to a collection container into which withdrawn fluid is collected. In some examples, aspiration suction may be facilitated by one or more pumps rather than a vacuum. Furthermore, in some embodiments, aspiration is primarily passive, rather than through active suction. Therefore, it should be understood that embodiments of the present disclosure may not include vacuum components.

As referenced above, the systems 150, 110, and 170 can include the control circuitry 401, 422, and 430, respectively, configured to perform certain functionality described herein. The term “control circuitry” can refer to any collection of one or more processors, processing circuitry, processing modules/units, chips, dies (e.g., semiconductor dies including one or more active and/or passive devices and/or connectivity circuitry), microprocessors, micro-controllers, digital signal processors, microcomputers, central processing units, graphics processing units, field programmable gate arrays, application specific integrated circuits, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry can further comprise one or more, storage devices, which can be embodied in a single memory device, a plurality of memory devices, and/or embedded circuitry of a device. Such data storage can comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware state machine (and/or implements a software state machine), analog circuitry, digital circuitry, and/or logic circuitry, data storage device(s)/register(s) storing any associated operational instructions can be embedded within, or external to, the circuitry comprising the state machine, analog circuitry, digital circuitry, and/or logic circuitry.

Although control circuitry is illustrated as a separate component from other components of the control system 150/robotic system 110/fluid management system 170, any or all of the other components of the control system 150/robotic system 110/fluid management system 170 can be embodied at least in part in the control circuitry. For instance, control circuitry can include various devices (active and/or passive), semiconductor materials and/or areas, layers, regions, and/or portions thereof, conductors, leads, vias, connections, and/or the like, wherein one or more of the other components of the control system 150/robotic system 110/fluid management system 170 and/or portion(s) thereof can be formed and/or embodied at least in part in/by such circuitry components/devices.

Further, although not illustrated in FIG. 4, one or more of the control system 150, the robotic system 110, and/or the fluid management system 170 can each include data storage/memory configured to store data/instructions. For example, data storage/memory can store instructions that are executable by control circuitry to perform certain functionality/operations. The term “memory” can refer to any suitable or desirable type of computer-readable media. For example, one or more computer-readable media can include one or more volatile data storage devices, non-volatile data storage devices, removable data storage devices, and/or nonremovable data storage devices implemented using any technology, layout, and/or data structure(s)/protocol, including any suitable or desirable computer-readable instructions, data structures, program modules, or other types of data. One or more computer-readable media that can be implemented in accordance with embodiments of the present disclosure includes, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

In some instances, the control system 150 and/or the robotic system 110 is configured to implement one or more localization techniques to determine/track an orientation/position of an object/medical instrument. For example, the one or more localization techniques can process input data to generate position/orientation data for a medical instrument. Position/orientation data of an object/medical instrument can indicate a position/orientation of the object/medical instrument relative to a frame of reference. The frame of reference can be a frame of reference relative to anatomy of a patient, a known object (e.g., an EM field generator, system, etc.), a coordinate system/space, and so on. In some implementations, position/orientation data can indicate a position/orientation of a distal end of a medical instrument (and/or proximal end, in some cases). For example, position/orientation data for a scope can indicate a position and orientation of a distal end of the scope, including an amount of roll of the distal end of the scope. A position and orientation of an object can be referred to as a pose of the object.

Example input data that can be used to generate position/orientation data for an object/medical instrument can include: sensor data from a sensor associated with a medical instrument (e.g., EM field sensor data, vision/image data captured by an imaging device/depth sensor/radar device on the medical instrument, accelerometer data from an accelerometer on the medical instrument, gyroscope data from a gyroscope on the medical instrument, satellite-based positioning data from a satellite-based sensor (a global positioning system (GPS), for example), and so on); feedback data from a robotic arm/component (also referred to as “kinematics data”) (e.g., data indicating how a robotic arm/component moved/actuated); robotic command data for a robotic arm/component (e.g., a control signal sent to the robotic system 110/robotic arm 112 to control movement of the robotic arm 112/medical instrument); shape sensing data from a shape sensing fiber (which can provide information regarding a location/shape of a medical instrument); model data regarding anatomy of a patient (e.g., a model of an interior/exterior portion of anatomy of the patient); position data of a patient (e.g., data indicating how the patient is positioned on a table); pre-operative data; etc.

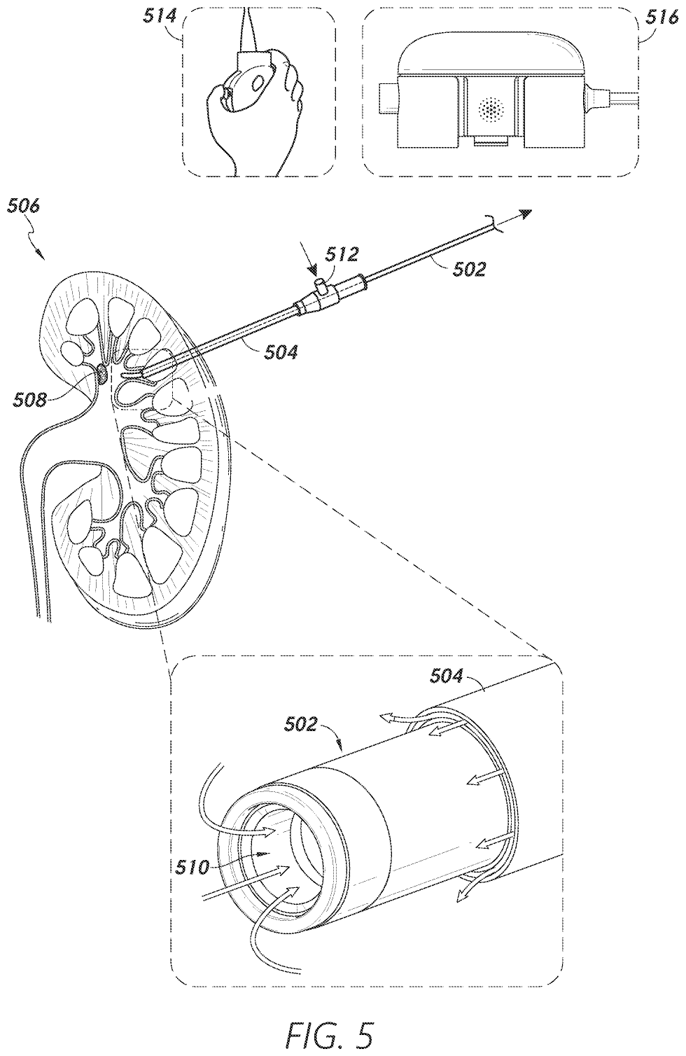

FIG. 5 illustrates an example catheter 502 and a percutaneous-access device 504 disposed at least partly in a kidney 506 of a patient in accordance with one or more embodiments. The catheter 502 and percutaneous-access device 504 may be representative any of the catheters and percutaneous-access devices discussed herein. In this example, the instruments 502, 504 are illustrated in the context of a urology procedure to treat/remove a kidney stone 508 from the kidney 506. However, the instruments 502, 504 can be used in other types of procedures. As noted above, urology procedures and/or other types of procedures can be implemented manually at least in part and/or can be performed using robotic technologies at least in part.

The catheter 502 can be configured to be articulated, such as with respect to at least a distal end/tip of the catheter 502. For instance, the distal end portion/tip of the catheter 502 can be deflected in a variety of directions. In examples, the catheter 502 can be configured to move with two degrees of freedom (2-DOF) (e.g., two of x, y, z, yaw, pitch, or roll movement). To illustrate, the distal end portion of the catheter 502 can be configured to move right/left or up/down (e.g., x, y, or z movement) and also move to insert/retract the catheter 502 (e.g., translate along the x, y, or z axis). In other examples, the catheter 502 can be configured to move with 3-DOF (e.g., three of x, y, z, yaw, pitch, or roll movement). To illustrate, the distal end portion of the catheter 502 can be configured to move right/left and up/down (e.g., two of x, y, or z movement) and also move to insert/retract the catheter 502. However, the catheter 502 can also be configured to move with 4-DOF (e.g., x, y, z, and pitch/yaw/roll movement), 6-DOF (e.g., x, y, z, pitch, yaw, and roll movement), and so on. In some embodiments, such as when the catheter 502 is implemented with a robotically-controllable handle, the catheter 502 is not configured for roll movements. However, the catheter 502 can be configured for roll and/or other types of movement in some cases, such as when the catheter 502 is configured with a manually-controllable handle or some robotically-controllable cases.

As shown, the catheter 502 can be implemented with the percutaneous-access device 504 to provide aspiration/irrigation to the kidney 506. The percutaneous-access device 504 may include one or more sheaths and/or shafts through which instruments (e.g., the catheter 502) and/or fluids may access the target anatomy in which the distal end of the device 504 is disposed. In some embodiments, active aspiration/suction may be drawn through a lumen 510 of the catheter 502 to a proximal end of the catheter 502 (e.g., a handle of the catheter 502). Further, in some embodiments, irrigation can be provided via the percutaneous-access device 504, such as between concentric sheaths. For example, a fluid management system (not illustrated) can be connected to an irrigation port 512 port to provide irrigation to the percutaneous-access device 504, which travels down the percutaneous-access device 504 to the target site. FIG. 5 illustrates an example of the flow of aspiration fluid into the lumen 510 of the catheter 502 and the flow of irrigation fluid from the percutaneous-access device 504. In some embodiments, a passive aspiration outflow channel may be formed in the space between the outer wall of the catheter 502 and an inner wall/sheath of the percutaneous-access device/assembly 504. When the catheter 502 is disposed within the percutaneous-access device 504, the catheter 502 and the shaft(s)/sheath(s) of the percutaneous-access device 504 may be generally concentric. The catheter 502 and the percutaneous access device 504 may have generally circular cross-sectional shapes over at least portions thereof.

The catheter 502 may be controllable in any suitable or desirable way, either based on manual control and/or robotic control. In FIG. 5, handles/bases 514, 516 provide examples that may be used to control the catheter 502. The handle 514 illustrates a hand-held/manual handle that is configured to be manipulated by a physician/user to control movement of the catheter 502. Meanwhile, the handle 516 illustrates a robotically controllable handle that is configured to be manipulated by a robotic arm, such as an end effector of a robotic arm, to control movement of the catheter 502. Example robotically-controllable and manually-controllable catheters are discussed in further detail below. By implementing an articulable catheter, the techniques/structures can allow various positions within the patient to be reached in a manner that prevents/minimizes damage to the anatomy of the patient. For example, a physician can navigate the distal portion of the catheter 502 to reach a particular cavity in the kidney 506 (e.g., calyx) where a kidney stone is located, without repositioning the rest of the shaft of the catheter 502 and/or the percutaneous-access device 504.

In embodiments, the catheter 502 is free of an imaging device. That is, the catheter 502 is implemented without an imaging device/camera on a distal end to capture image data of an internal anatomy of the patient. However, in other embodiments the catheter 502 can include an imaging device(s), such as on the tip of the catheter 502. Further, in embodiments, the catheter 502 is implemented without a position sensor (i.e., does not include a position sensor). However, the catheter 502 can be implemented with a position sensor in some cases, such as on a distal end of the catheter 502.

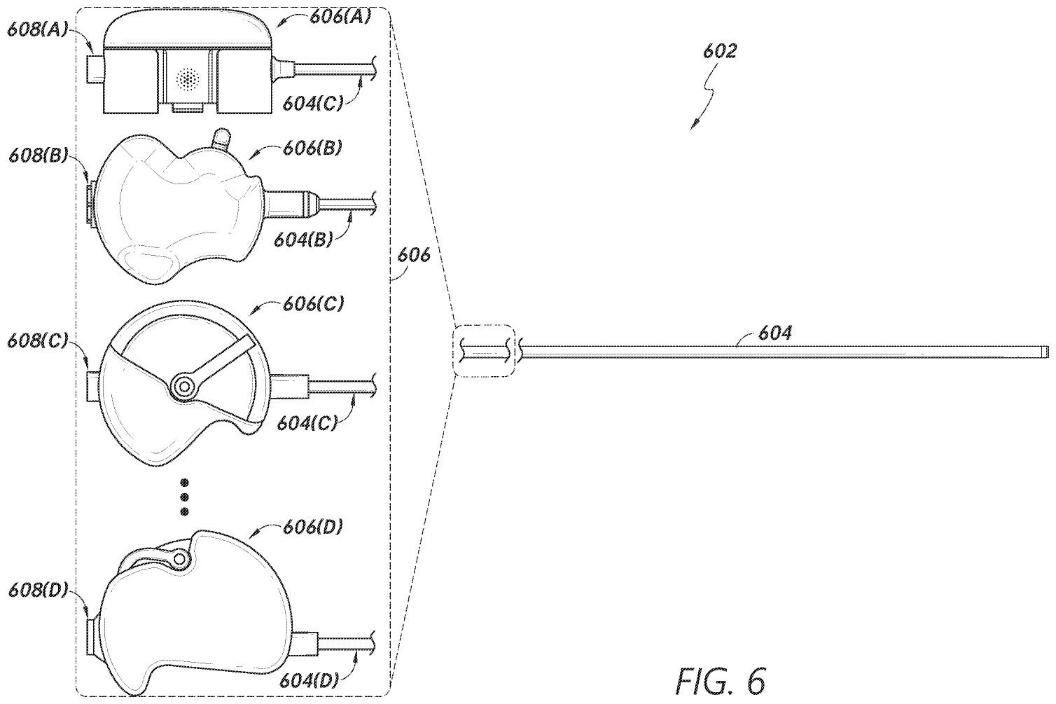

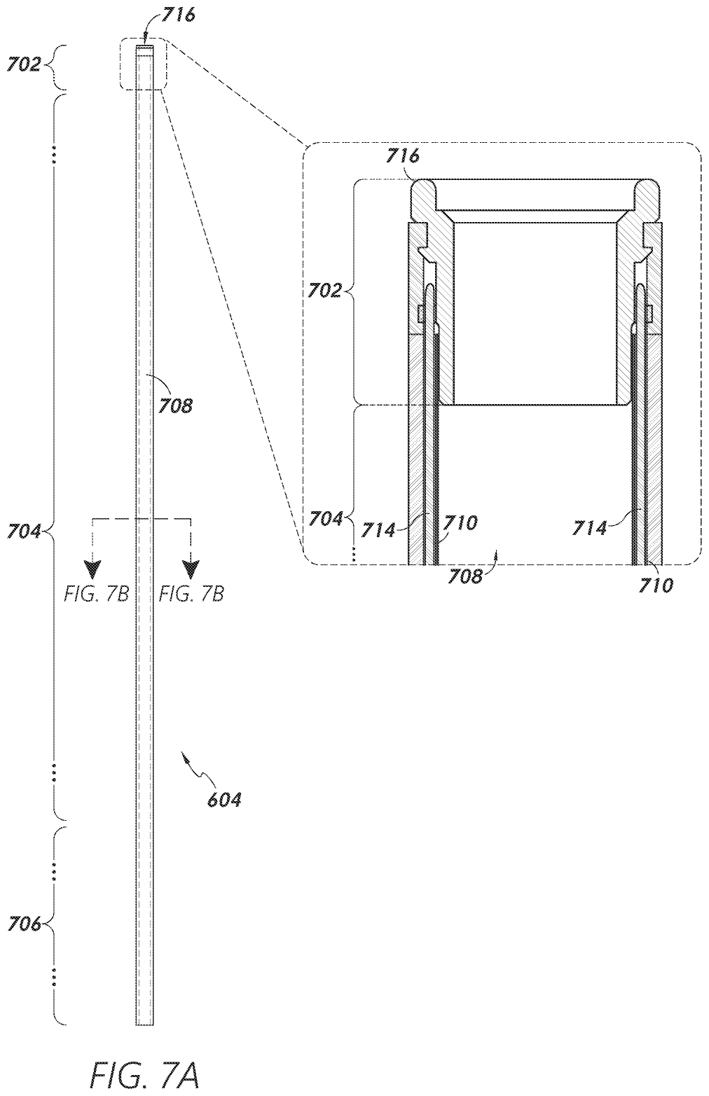

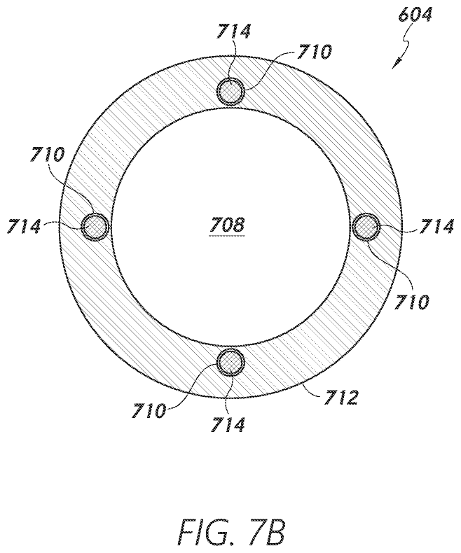

FIGS. 6 and 7 illustrates example features of a robotically/manually controllable catheter 602 in accordance with one or more embodiments of the present disclosure. The features of the catheter 602 may be implemented in the context of one or more of the catheters discussed herein. The catheter 602 includes an elongate shaft 604 connected to a handle/base 606 (also referred to as the “instrument base 606”) that is configured to control actuation of at least a portion of the elongate shaft 604. As shown in FIG. 6, the handle 606 can be implemented as a robotically controllable handle (e.g., the handle 606(A)) configured to couple to a robotic arm and/or a manually controllable handle (e.g., the handles 606(B), 606(C), and 606(D)) configured to be held/manipulated by a user. In some embodiments, the elongate shaft 604 can extend through the handle 606 to a port 608 of the handle 606, which can be connected to a fluid management system and/or another system to facilitate aspiration, irrigation, deployment of an instrument through a working channel of the catheter 602, and so on. Although certain handles are discussed in the context of being implemented in a manually-controllable catheter or robotically-controllable catheter, such catheters can be implemented in other contexts. For example, a manually-controllable catheter can include robotic components to be implemented as a robotically-controllable catheter (e.g., secondary use as a robotic catheter), and/or a robotically-controllable catheter can include manual component to be implemented as a manually-controllable catheter (e.g., secondary use as a manual catheter). As such, in some cases, a catheter is configured for both manual and robotic manipulation.

As shown in FIGS. 7A and 7B (and other figures), the shaft 604 can include a distal/tip section/portion 702 (sometimes referred to as “the distal end portion 702”), a middle/medial section/portion 704, a proximal section/portion 706 (sometimes referred to as “the proximal end portion 706”), and/or a lumen 708 that extends through at least a portion of the shaft 604. For example, the lumen 708 can extend through an entirety of the shaft 604 from the distal section 702 (that may be positioned at a target site in a patient) to the proximal section 706 (that may be connected to the port 608 of the handle 606). However, the lumen 708 can extend another distance through the catheter 602. In examples, the lumen 708 can be referred to as a working channel. The distal section 702, the middle section 704, and/or the proximal section 706 can each be implemented with any longitudinal length. The terms distal, middle/medial, proximal, and/or other terms are used to describe a position of a feature relative to another feature. For example, a proximal feature of the catheter 602 can refer to a feature that is farthest from a target or anatomical site (e.g., during use/a procedure), whereas a distal feature of the catheter 602 can refer to a feature that is closest to the target or anatomical site.

In some embodiments, the distal section 702 of the shaft 604 can include a filter/containment structure/feature 716 (also referred to as “the tip structure 716”) configured to prevent certain objects from entering into the rest of the shaft 604 and/or configured to contain an object at a distal end of the shaft 604, such as when aspirating through the shaft 604. For example, in the context of a urological procedure, the distal portion 702 of the catheter 602 can be positioned at a target site and used to aspirate one or more kidney stone fragments from a kidney. Here, the tip structure 716 can be configured to hold the kidney stone while the stone is being fragmented into pieces, such as by an instrument deployed from another device at the target site. The tip structure 716 can also prevent fragments that are larger than a particular size from being sucked into the rest of the shaft 604, which could clog the shaft 604 and impede/stop aspiration flow. Although the tip structure 716 can be implemented as a separate component from the rest of the shaft 604, the tip structure 716 can be integral with rest of the shaft 604 or implemented in other manners.

In instances where the tip structure 716 is implemented as a separate component from the rest of the shaft 604, the tip structure 716 can be attached to the rest of the shaft 604 with an adhesive, fastener, interlocking mechanism (e.g., tabs, grooves, etc.), and so on. In some embodiments, the shaft 604 includes a ring portion 1102 (as shown in FIG. 11 and elsewhere) to facilitate coupling of the tip structure 716 to the rest of the shaft 604 and/or to cover the tip structure 716 once the tip structure 716 is secured to the rest of the shaft 604. In this example, the shaft 604 (including the tip structure 716) are implemented in a substantially cylindrical form (e.g., having a circular cross-section); however, the shaft 604 can be take in other forms, such as a rectangular/square form or another shape.

In some embodiments, at least a portion of the shaft 604 can be formed of various materials, such as plastics, rubbers, vertebrae links, metal or plastic braids/coils, and so on, such that at least a portion of the shaft 604 is flexible for articulation. In some embodiments, the shaft 604 includes reinforcement material (e.g., braided) to strengthen and/or facilitate flexibility of the shaft 604. For example, the shaft 604 can include braid reinforcement for hoop strength and or to prevent kinking of the shaft 604 when the shaft 604 is navigated within the anatomy of a patient. Further, in some embodiments, the shaft 604 includes multiple layers of material that are implemented in a variety of configurations to facilitate the features of the shaft 604 discussed herein. In some cases, the tip structure 716 is formed of a different material than the rest of the shaft 604. For example, the tip structure 716 can be implemented with a material that avoids degradation in certain contexts, such as catastrophic degradation. The tip structure 716 can be implemented with stainless steel (or other types of steel), titanium, tungsten, and/or other materials (which may have relatively high melting points above a threshold) that can generally maintain its structure when laser beams inadvertently and/or occasionally contact the tip structure 716. However, the tip structure 716 and/or any other portion of the shaft 604 can be implemented with other materials.

The shaft 604 can include one or more lumens 710 (also referred to as “the one or more wire lumens 710”) disposed in a wall 712 of the shaft 604, such as an outer wall, as shown in the cross-sectional view of FIG. 7B taken along the line shown in FIG. 7A. The one or more lumens 710 can be spaced equidistantly apart around the wall of the shaft 604 or at another location. The catheter 604 can include one or more elongate movement members 714 slidably disposed in the one or more wire lumens 710. The one or more elongate movement members 714 can include one or more pull wires, cables, fibers, and/or flexible shafts. The one or more elongate movement members 714 can include any suitable or desirable materials, such as metallic and non-metallic materials, including stainless steel, Kevlar, tungsten, carbon fiber, and the like. In some embodiments, the catheter 602 is configured to exhibit nonlinear behavior in response to forces applied by the one or more elongate movement members 714. The nonlinear behavior may be based on stiffness and/or compressibility of the catheter 602, as well as variability in slack or stiffness between different elongate movement members 714. Although a particular number of wire lumens 710 and elongate movement members 714 are illustrated in the figures, any number of lumens and/or elongate movement members can be implemented.

The one or more elongate movement members 714 can be attached/extend to the distal section 702 of the shaft 604. At a proximal side, the one or more elongate movement members 714 can be coupled to a component(s) of the handle 606 (e.g., an input assembly) that is configured to control articulation of the shaft 604, such as by deflecting the distal section 702 of the shaft 604. The handle 606 can be configured to pull (and/or release tension of) the one or more elongate movement members 714 within the one or more lumens 710 to cause the distal section 702 to deflect from a longitudinal axis. In some embodiments, the catheter 602 is configured to move in two directions based on manipulation of the one or more elongate movement members 714 (e.g., up/down or right/left). In other embodiments, the catheter 602 is configured to move in four directions based on manipulation of the one or more elongate movement members 714 (e.g., up/down and right/left). In yet other embodiments, the catheter 602 is configured to move in other directions. In some robotic examples, the catheter 602 can move in any direction by using a combination of four primary directions and four elongate movement members.

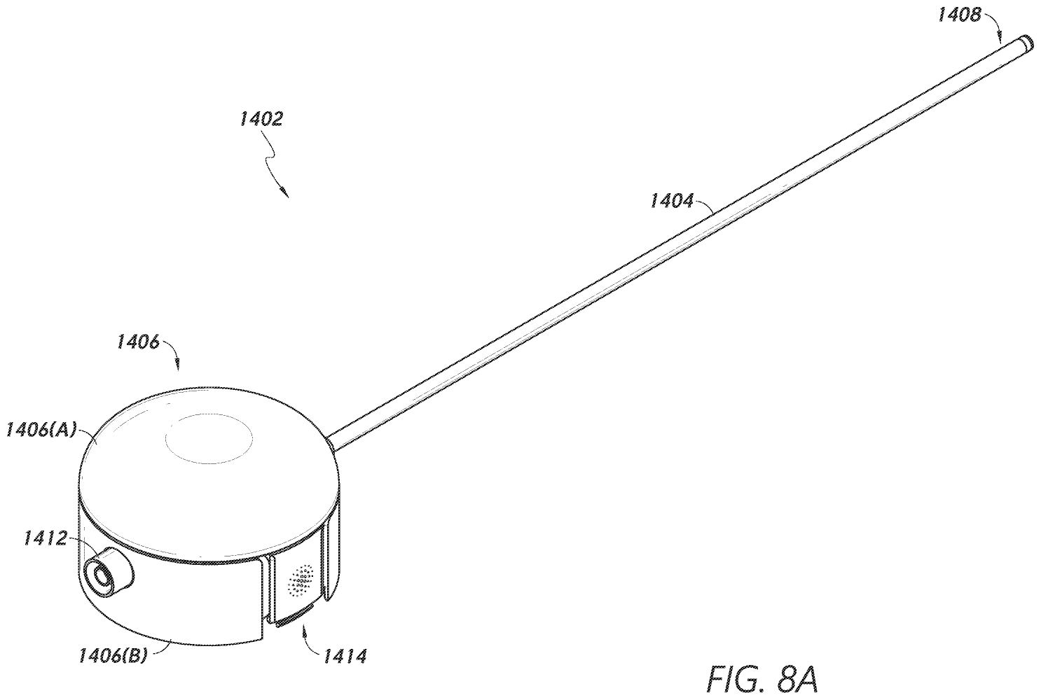

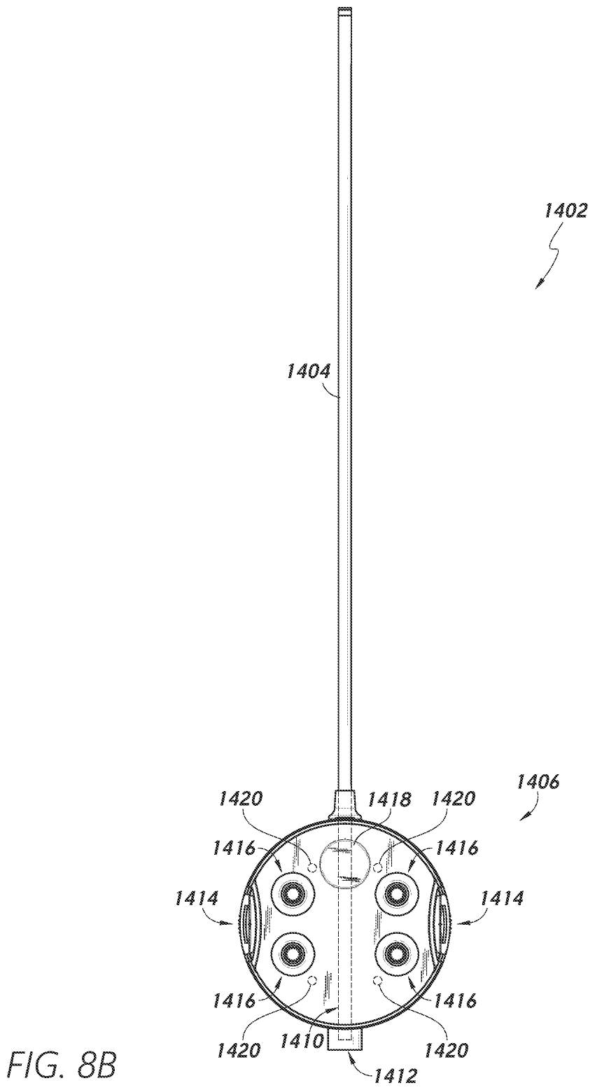

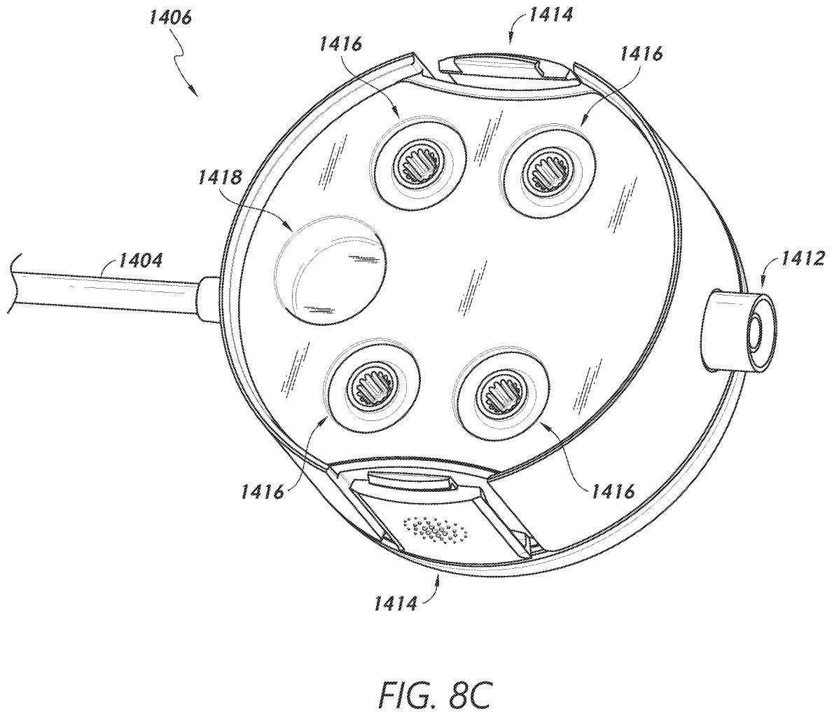

FIGS. 8-11 illustrate an example robotically-controllable catheter 1402 (sometimes referred to as “the robotically-controllable catheter assembly 1402”) in accordance with one or more embodiments of the present disclosure. In particular, FIG. 8A illustrates a perspective view of the catheter 1402, FIG. 8B illustrates a bottom view of catheter 1402, and FIG. 8C illustrates a perspective view of a bottom of the catheter 1402. As shown in FIGS. 8A-8C, the catheter 1402 includes an elongate shaft 1404 coupled to a handle/base 1406 (also referred to as the “instrument base 1406”) that is configured to control actuation of at least a portion of the elongate shaft 1404. The shaft 1404 can be representative of any of the shafts discussed herein. For example, the shaft 1404 can include a distal end portion 1408 configured to be disposed within a patient, a proximal end portion 1410 configured to couple to a port 1412 on the instrument base 1406, and a lumen (not illustrated) extending between the distal end portion 1408 and the proximal end portion 1410. The port 1412 can be configured to couple to a fluid management system, such as via an aspiration channel/tube. The port 1412 can protrude away from a surface of the instrument base 1406 and/or include other forms/structure to facilitate a connection with a channel/tube. Although the instrument base 1406 is illustrated with a substantially circular form, the instrument base 1406 can take other forms, such as a rectangular form.

The robotically-controllable catheter 1402 can include one or more attachment mechanisms 1414 configured to couple the instrument base 1406 to a robotic arm and/or another device/interface (e.g., a sterile adapter). The one or more attachment mechanisms 1414 can include one or more fasteners, such as clips, pins, hooks, buckles, clamps, screws, bolts, flanges, hook and loop, magnets, adhesives, and so on. The device/interface that is configured to couple to the one or more attachment mechanism 1414 can include one or more features to receive/connect to the one or more attachment mechanisms 1414. In some embodiments, the one or more attachment mechanisms 1414 are integral with a top portion 1406(A) of the instrument base 1406. However, the one or more attachment mechanisms 1414 can be implemented separately from the top portion 1406(A) or can be integrated into a bottom portion 1406(B) of the instrument base 1406 or otherwise implemented.

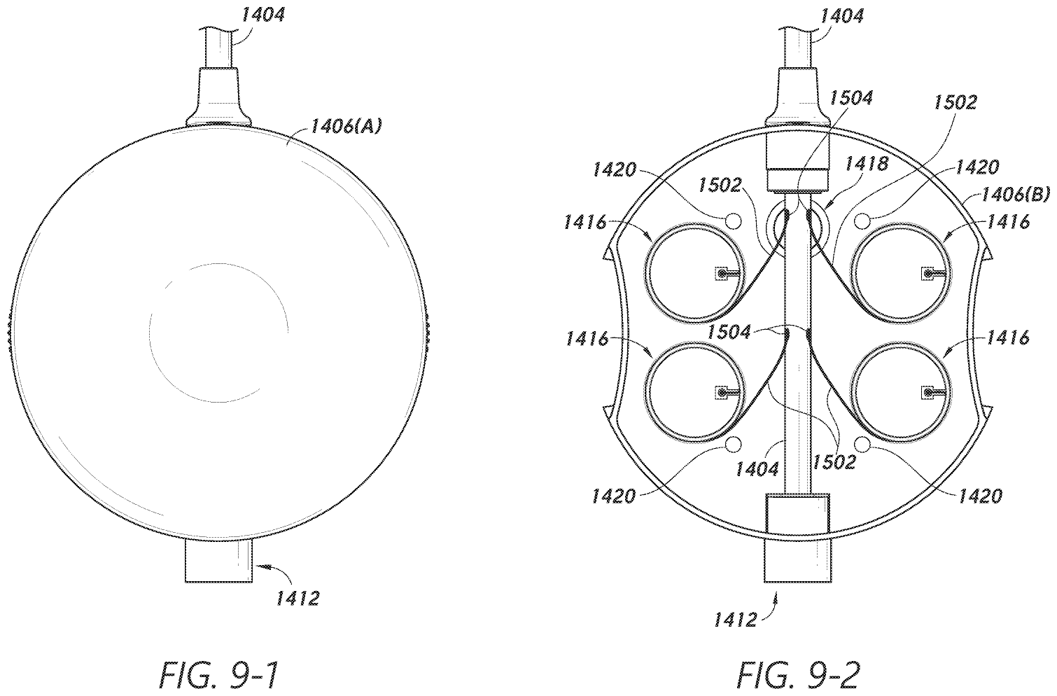

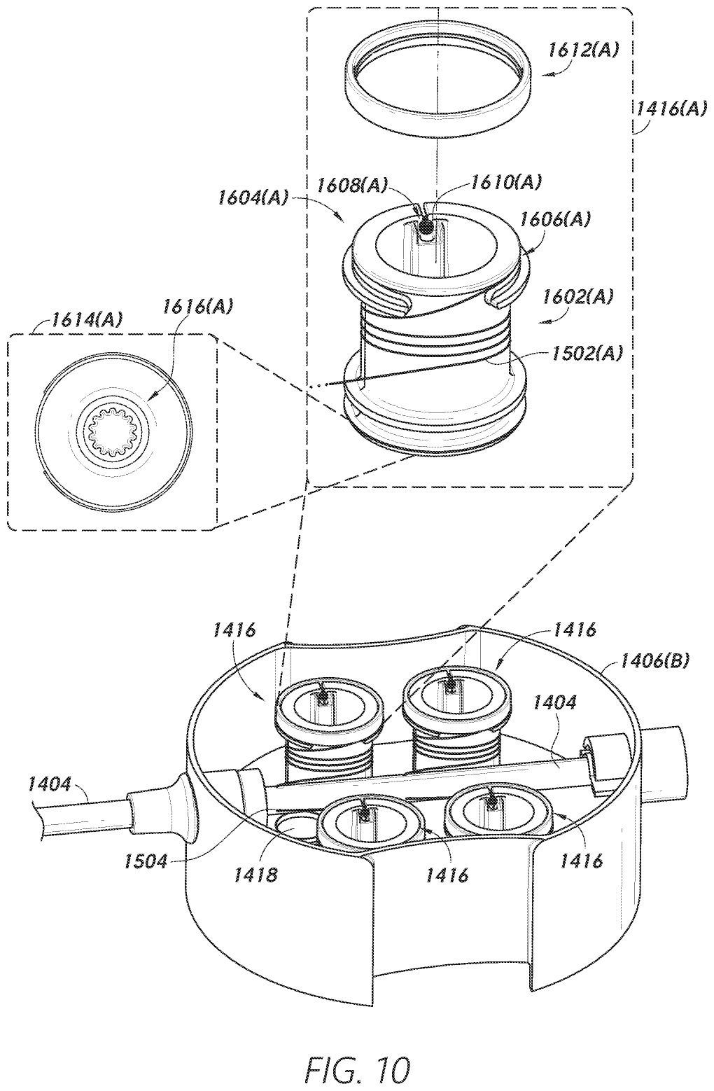

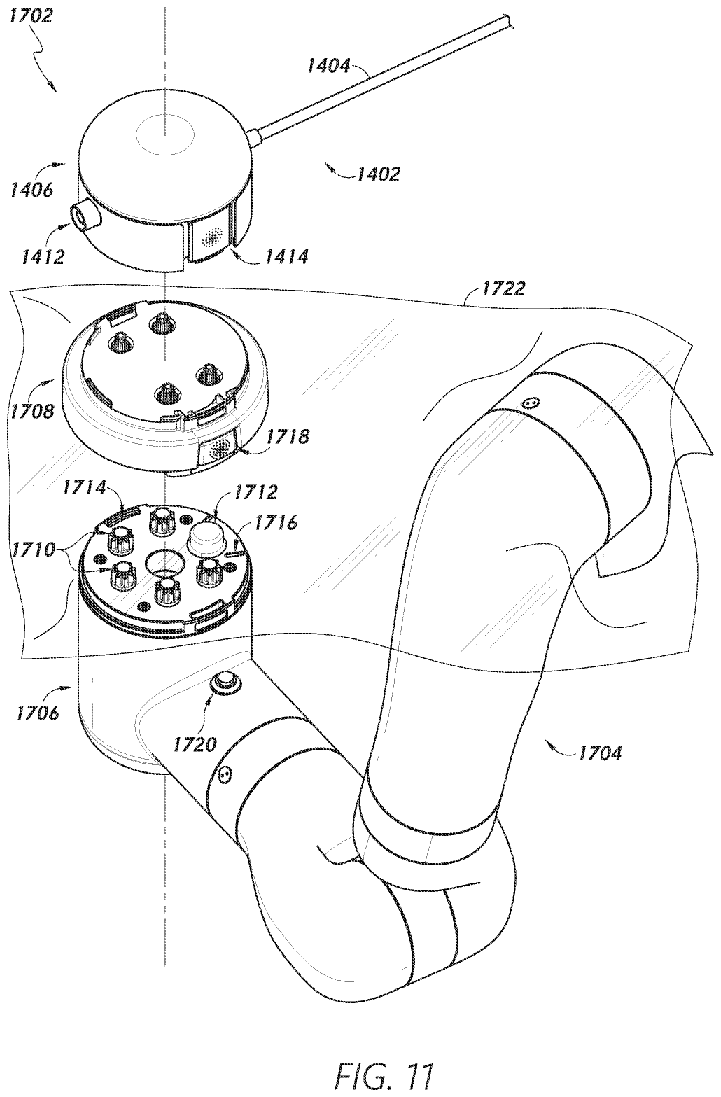

As shown in FIGS. 8-10, the robotically-controllable catheter 1402 can also include a drive input assembly 1416 configured to couple to a drive output assembly of a robotic arm and/or another device/interface. FIG. 9-1 illustrates the instrument base 1406 with the top portion 1406(A), while FIG. 9-2 illustrates the instrument base 1406 with the top portion 1406(A) removed to show the drive input assemblies 1416 and other features. A drive output assembly can interface with the drive input assembly 1416 to control articulation of the shaft 1404 of the catheter 1402. For example, the drive input assembly 1416 can be coupled to one or more elongate movement members 1502 (illustrated in FIGS. 9 and 10). The one or more elongate movement members 1502 can be slidably disposed within a portion of the shaft 1404 and attach to the distal end portion 1408 of the shaft 1404. The one or more elongate movement members 1502 can exit the shaft 1404 in the instrument base 1406 (e.g., towards the proximal end portion 1410) and couple to the drive input assembly 1416 within the instrument base 1406. The one or more elongate movement members 1502 can exit the shaft 1404 via one or more holes 1504 in the outer wall of the shaft 1404. The drive output assembly can actuate (e.g., rotate) the drive input assembly 1416 to pull (and/or release tension of) the one or more elongate movement members 1502, resulting in actuation of the distal end portion 1408 of the shaft 1404. Although various examples illustrate a spline interface coupling where a series of teeth on the outer and inner diameters of the outputs and inputs mate, the drive output assembly and the drive input assembly 1416 can be coupled via any of a variety of teeth, protrusions, or other mating engagement features and arrangements.

In this example, the drive input assembly 1416 includes one or more pulleys/spools 1602 configured to couple to the one or more elongate movement members 1502. FIG. 10 illustrates example details of a drive input assembly 1416(A). Here, the elongate movement member 1502(A) (which is implemented as a wire, in this case) exits the shaft 1404 within the instrument base 1406 and winds arounds the spool 1602(A) to attach to the spool 1602(A) and/or to remove slack in the pull wire 1502(A). The pull wire 1502(A) can exit the shaft 1404 at the appropriate location to avoid contact with other internal components of the instrument base 1406. For example, the shaft 1404 can include the one or more holes 1504 in the outer wall of the shaft 1404 at a particular distance from the proximal end of the shaft 1404, such that the one or more pull wires 1502 can exit from one or more wire lumens in the outer wall of the shaft 1404 and attach to the one or more pulleys 1602 without interfering with other components of the instrument base 1406. The one or more pull wires 1502 can exit the shaft 1414 at the same or different distances relative to the proximal end of the shaft 1404.

At a top end 1604(A) of the spool 1602(A), the pull wire 1502(A) can wrap into a channel/groove 1606(A) and can be secured/anchored at a distal end of the pull wire 1502(A) to a cavity 1608(A) using a stopper/enlargement/end feature 1610(A). However, other types of attachment mechanisms can be used, such as any type of fastener, adhesive, sandwiching/pinching the wire, soldering a metal ball at an end to create an anchor, laser melting an end into a ball shape that can be used as an anchor, etc. In this example, a ring 1612(A) is placed over the top end 1604(A) to maintain/secure the pull wire 1502(A). The pull wire 1502(A) can be coupled to the spool 1602(A) due to friction of the pull wire 1502(A) to the spool 1602(A), tension of the pull wire 1502(A), the stopper 1610(A), and/or the ring 1612(A). At a bottom end 1614(A) of the spool 1602(A), the spool 1602(A) can include a coupling mechanism/coupler 1616(A) configured to interface with a drive output assembly. For example, the coupling mechanism 1616(A) can include a gear or other mechanism. Although various example features are shown for the drive input assembly 1416, the drive input assembly 1416 can be implemented in a variety of other manners.

To control articulation of the shaft 1404, the one or more spools 1602/1416 can be rotated to pull (or release tension of) the one or more pull wires 1502 attached thereto. For example, rotating the spool 1602(A) in a counterclockwise direction with respect to FIG. 10 can cause the pull wire 1502(A) to more fully wraparound the spool 1602(A), resulting in a pull motion of the pull wire 1502(A). As such, the spool 1602(A) can be rotated to control an amount of slack/tension in the pull wire 1502(A). In some examples, multiple spools are rotated at the same time (e.g., in a cooperative manner) to facilitate articulation of the shaft 1404 in a particular direction. The spools can be rotated in the same or different directions to facilitate a particular movement. As noted above, a drive output assembly, such as that illustrated in FIG. 11, can control rotation of the one or more spools. In some embodiments, the catheter 1402 is configured to move in two directions, such as up and down or left, and right, based on manipulation of the one or more elongate movement members 1502. In other embodiments, the catheter 1402 is configured to move in four directions, such as up, down, left, and right, based on manipulation of the one or more elongate movement members 1502. In any event, the catheter 1402 can also be configured to be inserted/retracted, such as along a virtual rail, based on movement of the instrument base 1406 (e.g., movement of a robotic arm attached to the instrument base 1406).

In some embodiments, the robotically-controllable catheter 1402 can include a radio-frequency identification (RFID) tag 1418 and/or one or more other elements 1420 to facilitate calibration and/or identification of the catheter 1402, as shown in various figures. For example, the RFID tag 1418 can provide information to an RFID reader located on an instrument driver device (e.g., a robotic arm, sterile adapter, etc.) that is configured to connect to the instrument base 1406. The RFID reader can be configured to wirelessly obtain/read data from the RFID tag 1418 to calibrate the catheter 1402. The one or more elements 1420 can include one or more magnets, one or more Quick Response (QR) codes, one or more bar codes, and the like. In examples, a placement/location of one or more magnets 1420 on the instrument base 1406, a magnetic polarity, and/or a magnetic field strength of the one or more magnets 1420 can indicate a type of device. For example, an instrument driver device (that couples to the instrument base 1406) can be configured to detect a placement, magnetic field strength, and/or magnetic polarity of the one or more magnets 1420 and determine a type of device coupled to the instrument driver device based on such information (e.g., with two magnets in a device, identifying four devices: North North, North South, South North, and South South). Further, in some examples, a visual/optical system can scan one or more bar codes/QR codes 1420 to identify the type of device coupled to the instrument driver. In some embodiments, the RFID tag 1418 and/or the one or more elements 1420 are referred to as an identification element. Although the RFID tag 1418 and the one or more elements 1420 are generally discussed in the context of providing calibration data and identification information, respectively, the RFID tag 1418 and/or the one or more elements 1420 can each provide calibration data and/or identification information.

FIG. 11 shows an exploded view of an example instrument device manipulator assembly 1702 associated with a robotic arm 1704 in accordance with one or more embodiments. The instrument manipulator assembly 1702 includes an instrument driver 1706 (e.g., end effector) associated with a distal end of the robotic arm 1704. The instrument manipulator assembly 1702 further includes the instrument handle/base 1406 associated with the catheter 1402. The instrument handle 1406 can incorporate electro-mechanical means for actuating the instrument 1402/shaft 1404. In this example, the instrument 1402 is described as an aspiration catheter, but the instrument 1402 may be any type of medical/surgical instrument. Description herein of upward-facing and downward-facing surfaces, plates, faces, components, and/or other features or structures may be understood with reference to the particular orientation of the device manipulator assembly 1702 shown in FIG. 11. That is, although the instrument driver 1706 may generally be configurable to face and/or be oriented in a range of directions and orientations, for convenience, description of such components herein may be in the context of the generally vertical facing orientation of the instrument driver 1706 shown in FIG. 11.

In some embodiments, the instrument device manipulator assembly 1702 further includes an adapter 1708 configured to provide a driver interface between the instrument driver 1706 and the instrument handle 1406. The adapter 1708 and/or the instrument handle 1406 may be removable or detachable from the robotic arm 1704 and may be devoid of any electro-mechanical components, such as motors, in some embodiments. This dichotomy may be driven by the need to sterilize medical instruments used in medical procedures and/or the inability to adequately sterilize expensive capital equipment due to their intricate mechanical assemblies and sensitive electronics. Accordingly, the instrument handle 1406 and/or adapter 1708 may be designed to be detached, removed, and/or interchanged from the instrument driver 1706 (and thus the system) for individual sterilization or disposal. In contrast, the instrument driver 1706 need not be changed or sterilized in some cases and/or may be draped for protection.

The adapter 1708 (sometimes referred to as “the sterile adapter 1708”) can include connectors to transfer pneumatic pressure, electrical power, electrical signals, mechanical actuation, and/or optical signals from the robotic arm 1704 and/or instrument driver 1706 to the instrument handle 1406. For example, the adapter 1708 can include a drive input assembly(s) to couple to a drive output assembly(s) 170 of the end effector 1706 and a drive output assembly(s) configured to couple to a drive input assembly(s) of the instrument handle 1406. The drive input assembly and drive output assembly of the adapter 1708 can be coupled together to transfer control/actuation from the instrument driver 1706 to the instrument handle 1406.

The instrument handle 1406 may be configured to manipulate the catheter 1402 using one or more direct drives, harmonic drives, geared drives, belts and pulleys, magnetic drives, and/or other manipulator means or mechanisms. The robotic arm 1704 can advance/insert or retract the coupled catheter 1402 into or out of the treatment site. In some embodiments, the instrument handle 1406 can be removed and replaced with a different type of instrument handle, such as to manipulate a different type of instrument.

The end effector 1706 (e.g., instrument driver) of the robotic arm 1704 can include various components/elements configured to connect to and/or align with components of the adapter 1708, handle 1406, and/or catheter 1402. For example, the end effector 1706 can include the drive output assembly(s) 1710 (e.g., drive splines, gears, or rotatable disks with engagement features) to control/articulate a medical instrument, a reader 1712 to read data from a medical instrument (e.g., radio-frequency identification (RFID) reader to read a serial number from a medical instrument and/or other data/information), one or more fasteners 1714 to attach the catheter 1402 and/or the adapter 1708 to the instrument driver 1706, and markers 1716 to align with an instrument that is manually attached to a patient (e.g., an access sheath) and/or to define a front surface of the device manipulator assembly 1702. The one or more fasteners 1714 can be configured to couple to one or more attachment mechanisms 1718 of the adapter 1708 and/or the one or more attachment mechanisms 1414 of the handle 1406. In some embodiments, the end effector 1706 and/or the robotic arm 1704 includes a button 1720 to enable an admittance control mode, wherein the robotic arm 1704 can be manually moved.

In some configurations, a sterile drape 1722, such as a plastic sheet or the like, may be disposed between the instrument driver 1706 and the adapter 1708 to provide a sterile barrier between the robot arm 1704 and the catheter 1402. For example, the drape 1722 may be coupled to the adapter 1708 in such a way as to allow for translation of mechanical torque from the driver 1706 to the adapter 1708. The adapter 1708 may generally be configured to maintain a seal around the actuating components thereof, such that the adapter 1708 provides a sterile barrier itself. The use of the drape 1722 coupled to the adapter 1708 and/or more other component(s) of the device manipulator assembly 1702 may provide a sterile barrier between the robotic arm 1704 and the surgical field, thereby allowing for the use of a robotic system associated with the arm 1704 in the sterile surgical field. The driver 1706 may be configured to be coupled to various types of sterile adapters that may be loaded onto and/or removed from the driver 1706 of the robotic arm 1704. With the arm 1704 draped in plastic, the physician and/or other technician(s) may interact with the arm 1704 and/or other components of the robotic cart (e.g., screen) during a procedure. Draping may further protect against equipment biohazard contamination and/or minimize clean-up after procedure.

Although the particular adapter 1708 shown in FIG. 11 may be configured for coupling with the catheter handle 1406, such as an aspiration catheter handle, adapters for use with device manipulator assemblies in accordance with aspects of the present disclosure may be configured for coupling with any type of surgical or medical device or instrument, such as an endoscope (e.g., ureteroscope), basketing device, laser fiber driver, or the like.

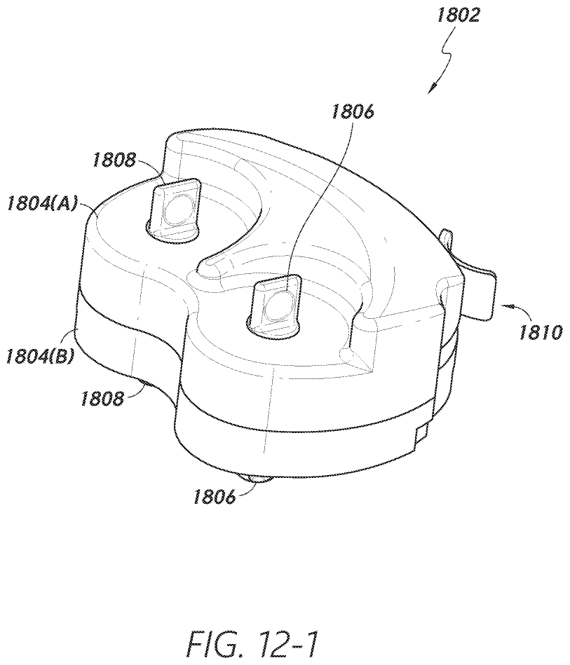

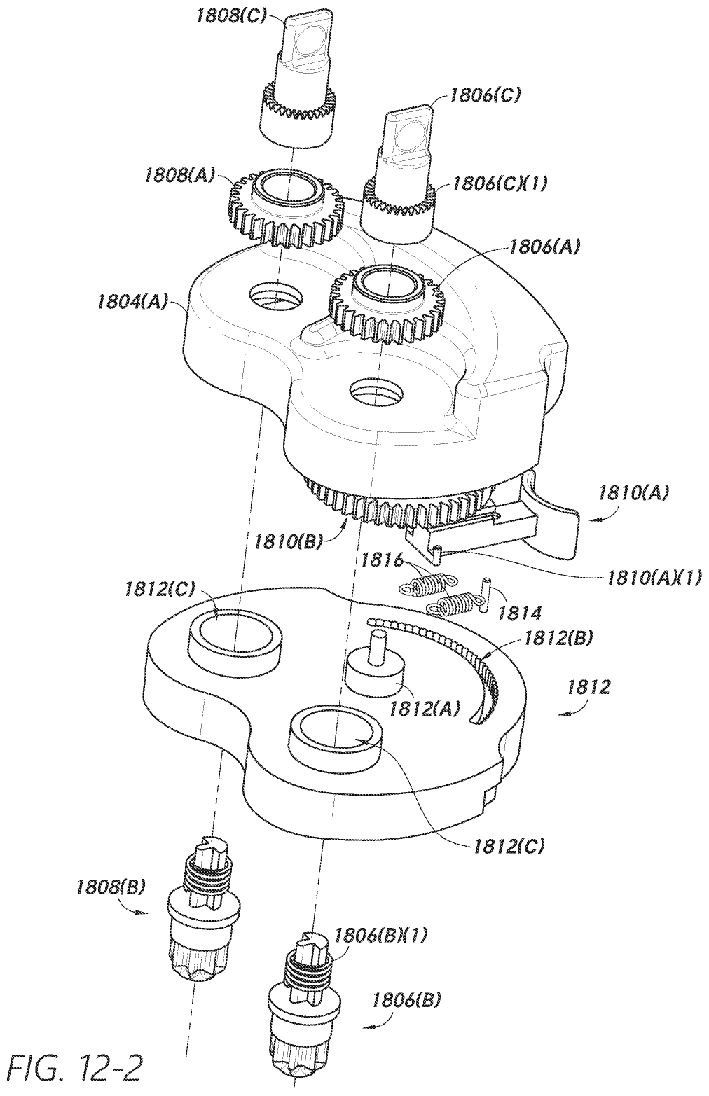

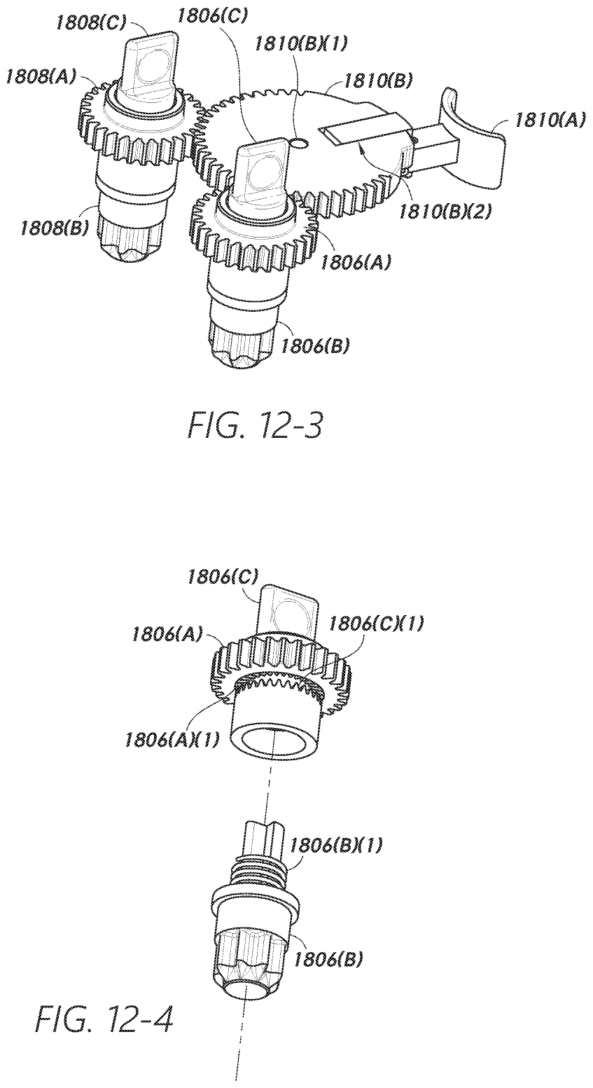

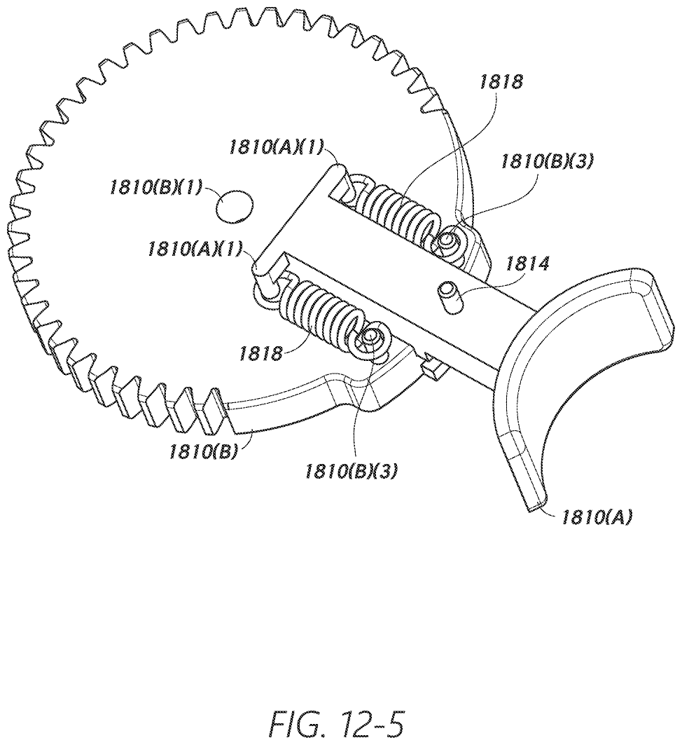

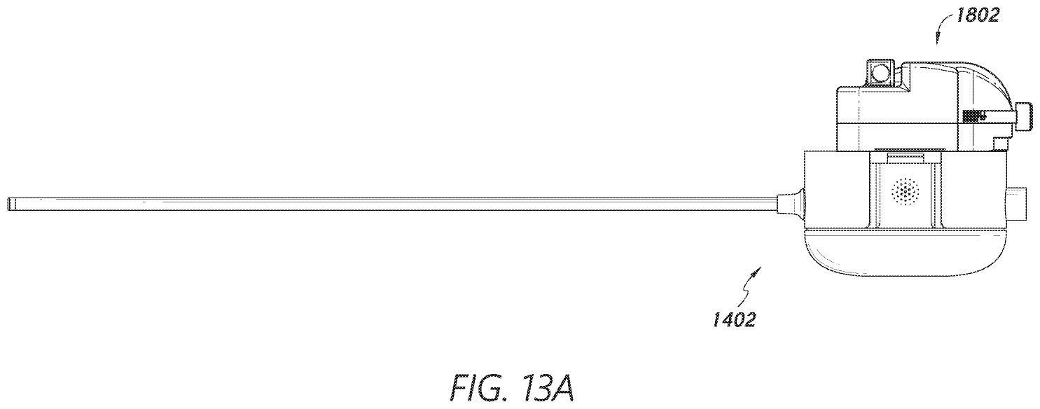

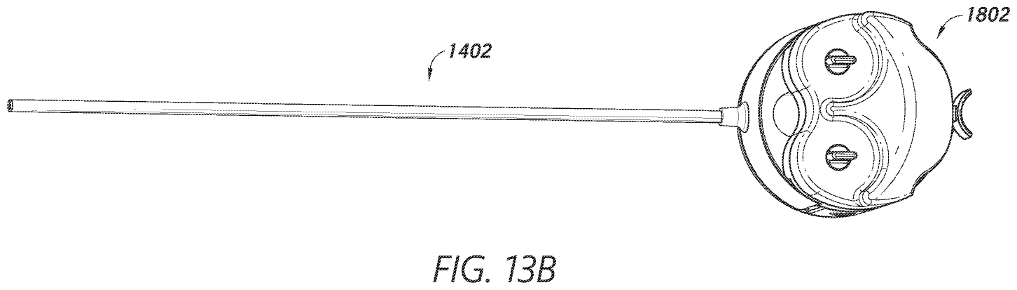

FIGS. 12 and 13 illustrate an example adapter 1802 (sometimes referred to as “the handheld instrument adapter 1802” or “manual adapter 1802”) configured to couple to a robotically-controllable medical instrument in accordance with one or more embodiments. The handheld instrument adapter 1802 can be configured to convert a medical instrument that is generally configured for robotic manipulation into a manually-controllable instrument. For example, the manual adapter 1802 can be configured to couple to a robotically-controllable medical instrument and receive manual input to control the robotically-controllable medical instrument in a manual manner, instead of using robotic controls. As such, in some embodiments, a medical instrument can be configured to operate in a robotic mode in which an instrument base is detached from the manual adapter 1802 and receives robotic input to control the medical instrument, such as articulation of a shaft of the medical instrument, and configured to operate in a manual mode in which the instrument base is coupled to the manual adapter 1802 and receives manual input to control the medical instrument.

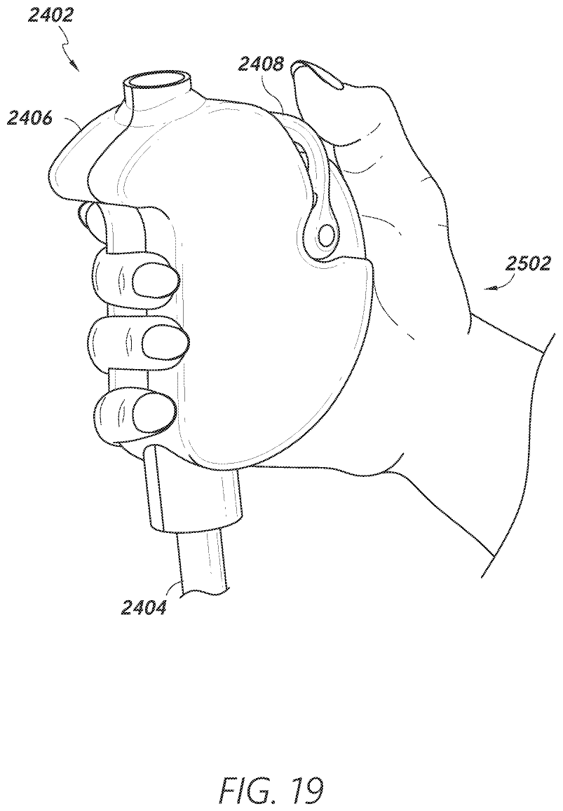

As shown in FIG. 12-1, the manual adapter 1802 can include a base/housing 1804, one or more couplers 1806, 1808 supported in the base 1804, and a manual actuator 1810 coupled to the couplers 1806, 1808. The couplers 1806, 1808 can be configured to couple to a drive input assembly of a robotically-controllable medical instrument. The manual actuator 1810 can be configured to manipulate the couplers 1806, 1808 to cause the robotically-controllable medical instrument (illustrated in FIG. 19) to articulate. For example, the manual actuator 1810 can cause one or more of the couplers 1806, 1808 to rotate, thereby rotating one or more components of a drive input assembly coupled to the couplers 1806, 1808. Although two couplers 1806, 1808 are illustrated, the couplers 1806, 1808 can include any number of couplers. In examples, the base 1804 includes a top portion 1804(A) and a bottom portion 1804(B); however, the base 1804 can be implemented in a variety of manners, such as a single piece.

As shown in FIGS. 12-2 through 12-4, the couplers 1806, 1808 can each include/attach to an engagement/disengagement assembly, which can be configured to engage/disengage the manual actuator 1810 from controlling a medical instrument. Each engagement assembly 1806, 1808 can allow for adjustment of the tension of one or more elongate movement members associated with the medical instrument. For example, the coupler/engagement assembly 1806 can include a first engagement/coupling member 1806(A) configured to engage with the manual actuator 1810, a second engagement member 1806(B) configured to engage with a drive input assembly of a medical instrument, and/or a manual actuator/tab 1806(C) configured to interface between the first engagement member 1806(A) and the second engagement member 1806(B). The tab 1806(C) can be configured to receive manual input to disengage the coupling of the first engagement member 1806(A) to the second engagement member 1806(B), as discussed in further detail below. In some embodiments, each engagement assembly 1806, 1808 is implemented as a gear assembly (e.g., one or more gears that are configured to engage/disengage with each other and/or a drive input assembly). Furthermore, although an engagement/disengagement assembly is shown in many figures, in some instances such feature is not implemented.

In the example shown, the second engagement member 1806(B)/1808(B) and the tab 1806(C)/1806(C) are keyed, so that the elements can interlock (e.g., avoid rotation of one element relative to the other when coupled together). Further, as shown in FIGS. 12-2 and 12-4, the tab 1806(C)/1808(C) and the first engagement member 1806(A)/1808(A) can be coupled via a gear or other mechanism. For example, the tab 1806(C) can includes a first gear 1806(C)(1) configured to engage with a second gear 1806(A)(1) on the first engagement member 1806(A). When disposed/installed in the base 1804 (e.g., in a use state), the second engagement member 1806(B) can hold/receive a spring 1806(B)(1) configured to exert a force (e.g., axial force) on the tab 1806(C) to engage the first gear 1806(C)(1) with the second gear 1806(A)(1). In this engaged state (e.g., a default/general use state), the first engagement member 1806(A), the second engagement member 1806(B), and the tab 1806(C) can rotate together in a direct correspondence. As such, the second engagement member 1806(B) can be coupled to the manual actuator 1810 indirectly such that movement of the manual actuator 1810 causes the second engagement member 1806(B) to rotate within the base 1804. Thus, the engagement assembly 1806 can be rotatably supported in the base 1804. FIG. 12-3 illustrates the elements operating in an engaged state where manual actuation of the manual actuator 1810 causes the second engagement member 1806(B)/1808(B) to rotate.

In some embodiments, the manual actuator 1810 can be disengaged from the second engagement member 1806(B)/1808(B), which can allow the second engagement member 1806(B)/1808(B) to rotate independently from the first engagement member 1806(A)/1806(A). This may be useful to provide manual input to rotate a drive input assembly of a medical instrument to adjust tension/slack in one or more elongate movement members of the medical instrument. For example, a user can press down on the tab 1806(C) to cause the first gear 1806(C)(1) of the tab 1806(C) to disengage from the second gear 1806(A)(1) of the first engagement member 1806(A). Since the tab 1806(C) and the second engagement member 1806(B) are coupled together (e.g., via a pairing), this can cause the second engagement member 1806(B) to decouple from the first engagement member 1806(A). Once such elements are decoupled/disengaged, the user can twist/rotate the tab 1806(C) to cause the second engagement member 1806(B) to rotate without affecting a position of the manual actuator 1810. Such rotation can ultimately result in adjustment to the tension in one or more elongate movement members of a medical instrument. This can allow a user to remove slack in the one or more elongate movement members (which can occur, such as in cases when the medical instrument is removed from a robotic arm). For example, the user can prepare/calibrate the adapter 1802 for use by removing slack in the one or more elongate movement members when the shaft of the medical instrument is straight and/or the manual actuator 1810 is positioned in a middle location. Thus, in some embodiments, one or more of the elements of the assembly 1806/1808 can be referred to as a “tensioning mechanism” and/or a “disengagement mechanism.”

For ease of discussion, the above example refers to certain example features of the engagement assembly 1806. It should be understood that the engagement assembly 1808 can operate in a similar fashion as the engagement assembly 1806. Furthermore, although the engagements assemblies 1806, 1808 are implemented with various gears in this example, the engagement assemblies 1806, 1808 can be implemented in other manners, such as with other mechanical mechanisms.

In some embodiments, the manual actuator 1810 includes an elongate member 1810(A) coupled to a gear/coupler 1810(B) that is configured to couple to/engage with the engagement assemblies 1806, 1808. As shown in FIG. 12-2, the adapter 1802 can include a plate 1812 having a pin/rotational feature 1812(A) to facilitate movement of the manual actuator 1810. For instance, the gear 1810(B) can be rotatably disposed on the plate 1812 with the pin 1812(A) extending into a hole 1810(B)(1) on the plate 1812. The gear 1810(B) can rotate on the pin 1812(A), resulting in rotation of the elongate member 1810(A). As shown, the plate 1812 can also include holes/recessed portions 1812(C) configured to receive/maintain the engagement assemblies 1806, 1808.

In some implementations, the manual actuator 1810 can be locked into place after movement of the manual actuator 1810. For example, a user can push down on the manual actuator 1810, move the manual actuator 1810 in a forward or backwards direction, and release the manual actuator 1810 to lock the manual actuator 1810 into place. In some examples, to facilitate such features, the elongate member 1810(A) can be connected to a pin 1814 that is configured to be placed in a groove 1812(B) that includes one or more teeth/recessed portions. The elongate member 1810(A) can move/slide within a groove 1810(B)(2) of the gear 1810(B) (as shown in FIG. 12-3) and can generally be forced outward away from the pivot point of the gear 1810(B). In this example, one or more springs 1816 (as shown in FIG. 12-5) are configured to couple to an end/attachment feature 1810(A)(1) of the elongate member 1810(A) and to an attachment feature 1810(B)(3) of the gear 1810(B). This can exert a force to pull the elongate member 1810(A) away from the pivot point of the gear 1810(B), and ultimately, pull the pin 1814 (attached to the elongate member 1810(A)) into the teeth in the groove 1812(B). A user can push down on the elongate member 1810(A) to cause the pin 1814 to release from the teeth and slide freely within the groove 1812(B). In a released state, the user can move/rotate the elongate member 1810(A) to another position. Although the locking feature is shown with particular elements, the locking feature can be implemented in a variety of other manners.

FIGS. 13A and 13B illustrate the manual adapter 1802 coupled to the robotically-controllable catheter 1402 in accordance with one or more embodiments. In such configuration, the robotically-controllable catheter 1402 can be controlled based on manual input provided by a user through the manual adapter 1802. Although FIGS. 13A and 13B illustrate the adapter 1802 coupled to the robotically-controllable catheter 1402, the adapter 1802 can be coupled to other types of robotically-controllable medical instruments, such as a robotically-controllable scope or another instrument, to enable the robotically-controllable medical instrument to be controlled using manual input.

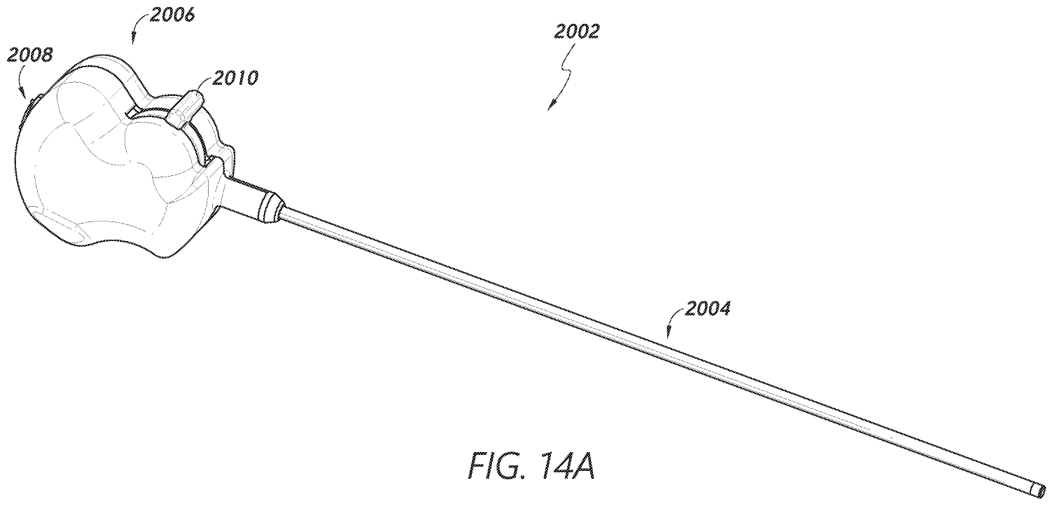

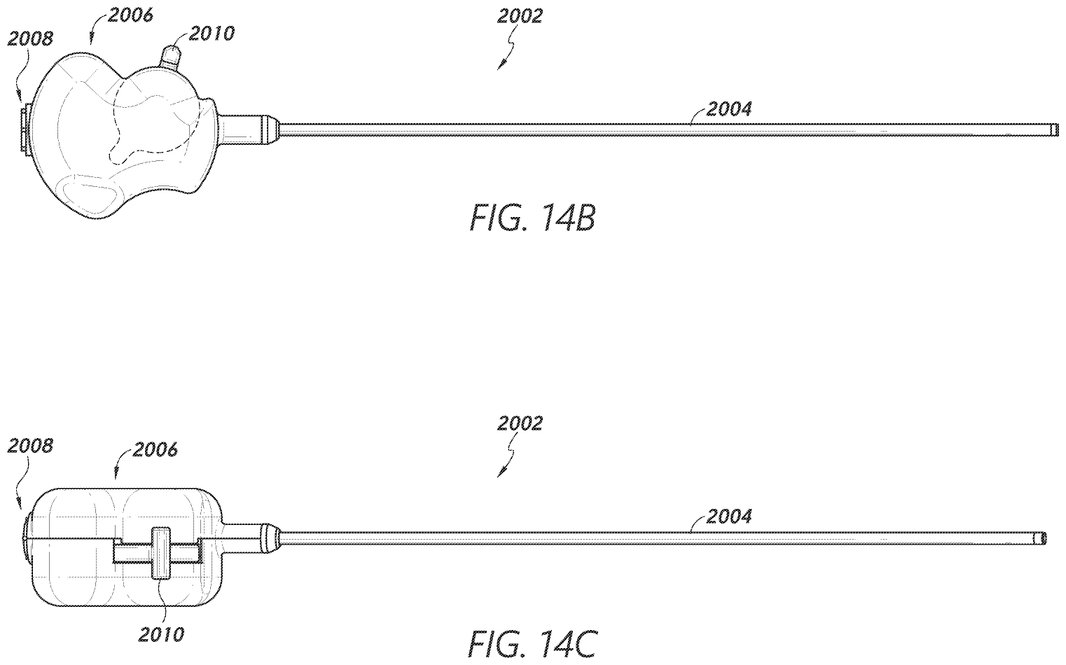

FIGS. 14-17 illustrate an example manually-controllable catheter 2002 in accordance with one or more embodiments of the present disclosure. As shown in FIGS. 14A-14C, the catheter 2002 includes an elongate shaft 2004 coupled to a handle/base 2006 that is configured to control actuation of at least a portion of the elongate shaft 2004. The shaft 2004 can be representative of any of the shafts discussed herein. For example, the shaft 2004 can include a distal end portion configured to be disposed within a patient, a proximal end portion configured to couple to a port 2008 on the handle 2006, and an aspiration/irrigation lumen (not illustrated) extending between the distal end portion and the proximal end portion. The port 2008 can be configured to couple to a fluid management system, such as via an aspiration channel/tube. The port 2008 can be removable from the handle 2006/shaft 2004 and/or integrated into the handle 2006/shaft 2004. As shown, the catheter 2002 can include a manual actuator 2010 configured to control actuation of the elongate shaft 2004. For example, the manual actuator 2010 can be configured to receive manual input from a user to control actuation of the distal end portion of the elongate shaft 2004.

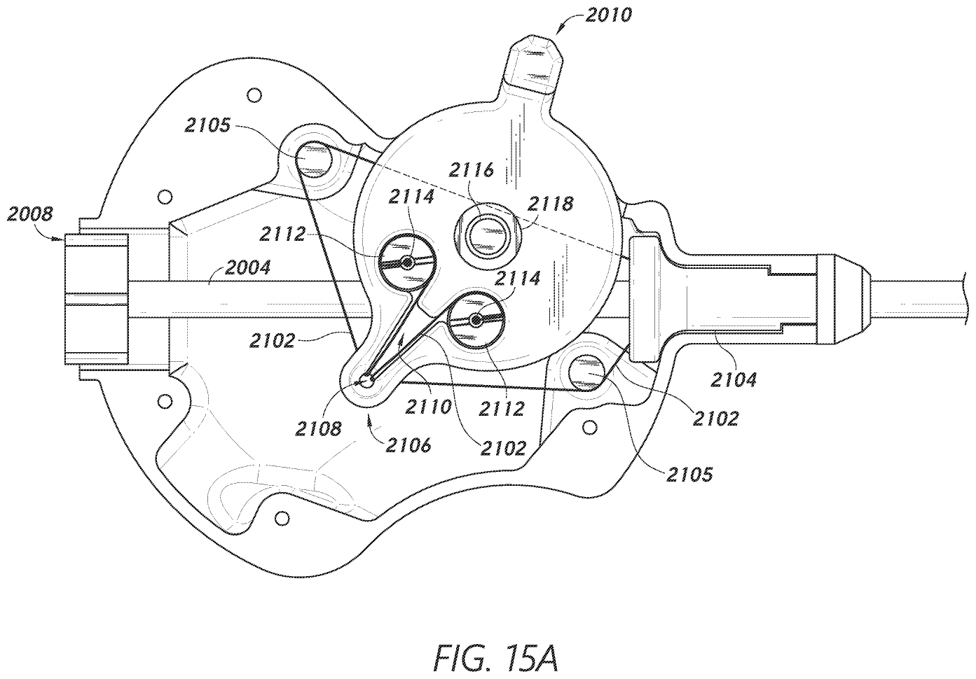

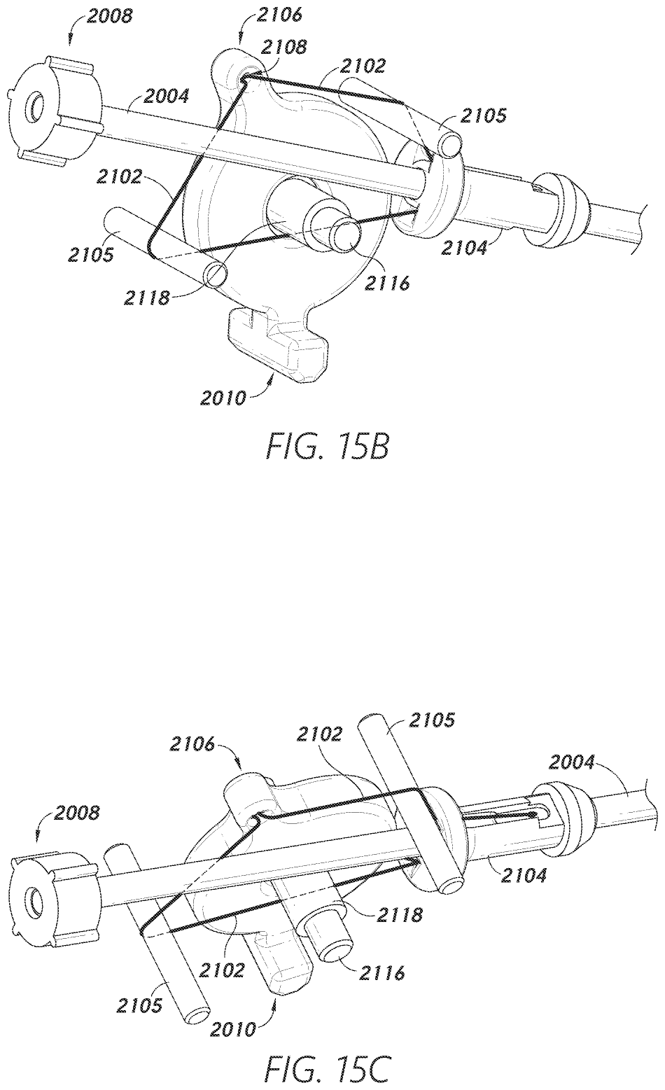

As shown in FIGS. 15A-15C, which illustrate the internal components of the handle 2006 (with the external enclosure partially removed in FIG. 15A and fully removed in FIGS. 15B and 15C), the manual actuator 2010 can be coupled to one or more elongate movement members 2102 (e.g., pull wires) that are at least partially disposed within the elongate shaft 2004. Here, the catheter 2002 includes two elongate movement members 2102 coupled to the distal end portion of the shaft 2004; however, any number of elongate movement members 2102 can be implemented. The elongate movement members 2102 can exit the wall of the elongate shaft 2004 within the handle 2006. In this example, the handle 2006 includes a guide/alignment structure 2104 located at a distal end of the handle 2006 to route the elongate movement members 2102 to the manual actuator 2010. The guide structure 2104 can include one or more grooves, openings, and so on. In some examples, such as that shown, the elongate movement members 2102 extend around one or more pins/shafts 2105 (which are supported/connected to the housing/enclosure) to route the elongate movement members 2102 to the manual actuator 2010. The manual actuator 2010 can include a substantially circular form with a protrusion/extension 2106 to receive the one or more elongate movement members 2102. The elongate movement members 2102 can be routed through a hole 2108 in the protrusion 2106. The protrusion 2106 can enable substantial movement of the distal end of the shaft 2004 when the manual actuator 2010 is actuated (e.g., more movement than if the protrusion 2106 were eliminated).

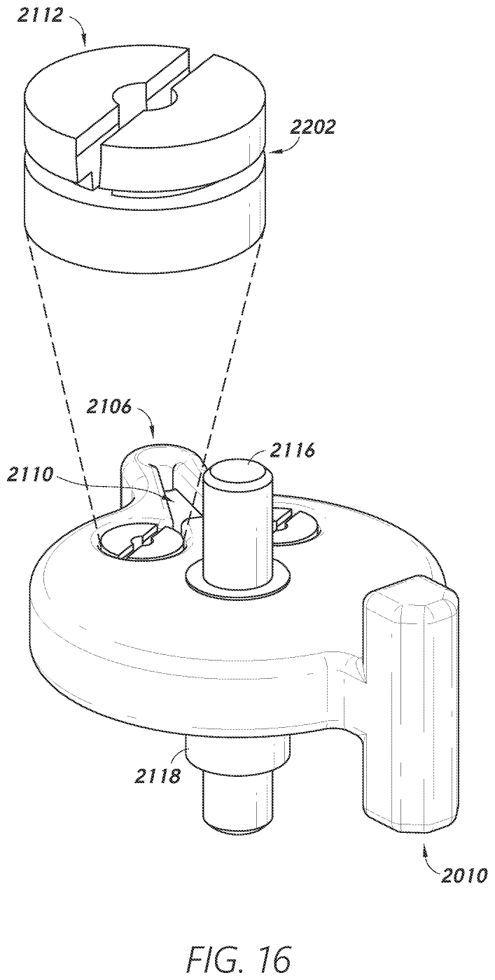

The manual actuator 2010 can also include a recess 2110 to align the elongate movement members 2102 with spools/anchoring members 2112. In some embodiments, the elongate movement members 2102 can wrap at least partially around the spools 2112 into grooves 2202, as shown in FIG. 16. The distal ends of the elongate movement members 2102 can be attached/anchored to the spools 2112 using a stopper/enlargement feature 2114, as shown in FIG. 15A. In examples, once the elongate movement members 2102 are wound around the spools 2112, the spools 2112 and/or the elongate movement members 2102 can be fixed to the manual actuator 2010 using an adhesive and/or a fastener. In some implementations of manufacturing/calibrating the catheter 2002, the manual actuator 2010 is placed in a middle position with respect to an available range of movement, and the spools 2112 are rotated to remove slack in the elongate movement members 2102 (with the shaft 2004 being positioned in a straight orientation, like that illustrated in FIGS. 14A-14C). The adhesive/fastener is then applied to secure the elongate movement members 2102 and/or the spools 2112 to the manual actuator 2010.

As shown in various figures, the manual actuator 2010 can be rotatably disposed/supported on a shaft 2116 and/or sleeve 2118 to facilitate rotation of the manual actuator 2010 with respect to the handle 2006. In particular, the manual actuator 2010 can include a hole in the center of the manual actuator 2010 (with respect to the substantially circular structure of the manual actuator 2010 as shown in FIG. 15A, for example) to receive the shaft 2116 and the sleeve 2118, with the shaft 2116 positioned within the sleeve 2118. The shaft 2116 and/or the sleeve 2118 can be coupled to the handle 2006, such as an external enclosure/housing of the handle 2006.

Although this example discusses various example features of the catheter 2002, other features can be implemented. For instance, the elongate movement members 2102 can be routed and/or attached to the manual actuator 2110 in other manners, such as by using other type of fastening/routing mechanisms. Further, the manual actuator 2010, the handle 2006, and/or other features can include different forms than those shown in FIGS. 14-17. In some instances, any of the components of other example catheters discussed herein (whether robotic or manual) can alternatively, or additionally, be implemented. Furthermore, any of the components of the catheter 2002 can be implemented in other catheters discussed herein.

In some embodiments, the manual catheter 2002 is configured to move in two directions, such as up and down or left and right, based on manipulation of the manual actuator 2010, such as manual input by a user. In examples, the distal and portion of the elongate shaft 2004 (and/or elongate shafts of other catheters) can articulate at least 150 degrees, 90-270 degrees, and so on with respect to a longitudinal axis of the elongate shaft 2004; however, various other degrees of movement can be implemented. The catheter 2002 can also be configured to be inserted/retracted and/or rolled based on movement of the handle 2006, such as by a user. In some instances, the catheter 2002 can include two elongate movement members 2102, with each elongate movement member 2102 attached to a different portion of the distal end portion of the shaft 2004, such as a top/left portion of a tip and a bottom/right portion of the tip. In use, actuation of the manual actuator 2010 can cause one of the elongate movement members 2102 to be pulled and tension of the other elongate movement member 2102 to be released. However, in other examples the catheter 2002 can be configured to move in more than two directions, such as up, down, left, and right, and/or the catheter 2002 can include more than two elongate movement members 2102 to facilitate such movement.

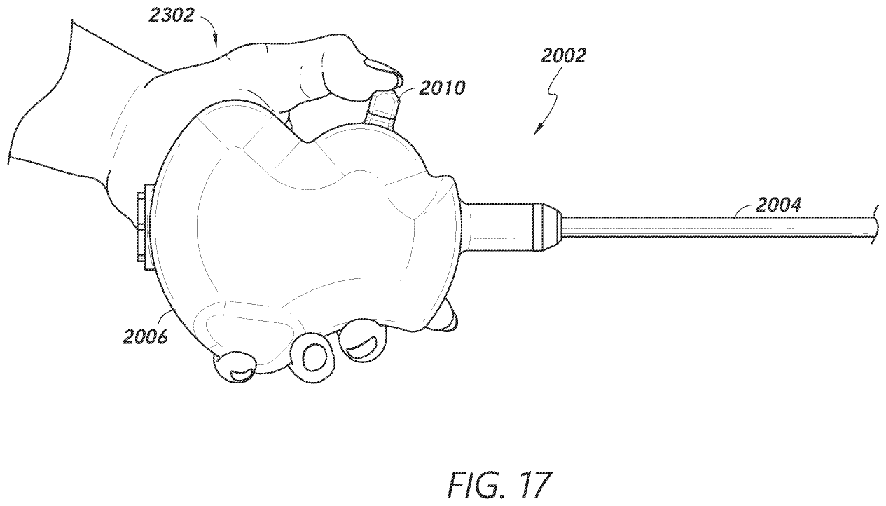

As shown in FIG. 17, in some implementations, the handle 2006 of the catheter 2002 is configured to be held/manipulated by a user 2302 in an underhand manner. Here, the user's thumb can contact/actuate the manual actuator 2010 and the rest of the user's fingers can grip around the handle 2006 to the opposite side of the handle 2006. The user 2302 can move his/her thumb in a forward or backwards direction (with respect to FIG. 17) to cause the manual actuator 2010 to articulate forward or backwards, resulting in articulation of the distal end portion of the elongate shaft 2004. However, the catheter 2002 can be held/manipulated by a user in a variety of other manners. In some examples, the user can roll the catheter 2002 by twisting his/her wrist.

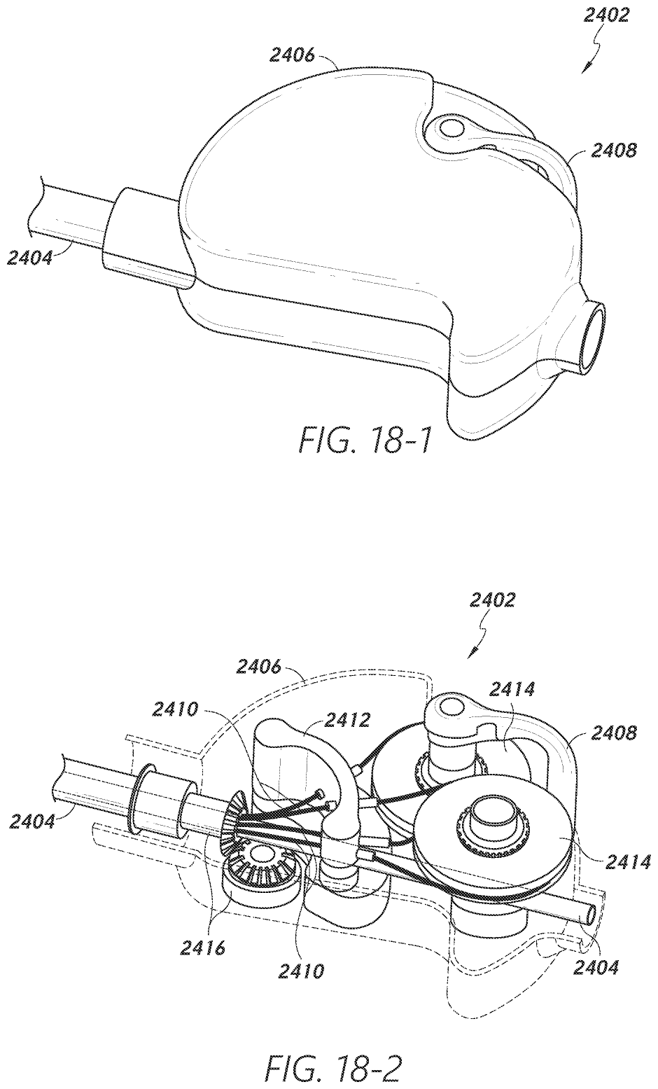

FIGS. 18-19 illustrate another example manually-controllable catheter 2402 in accordance with one or more embodiments of the present disclosure. As shown, the catheter 2402 includes an elongate shaft 2404 coupled to a handle/base 2406 that is configured to control actuation of at least a portion of the elongate shaft 2604. The shaft 2404 can include any of the shafts discussed herein. The catheter 2402 can include a manual actuator 2408 configured to control actuation of the elongate shaft 2404. For example, the manual actuator 2408 can be configured to receive manual input from a user to control actuation of the distal end portion of the elongate shaft 2404. The handle 2406 of the catheter 2402 can include one or more housing/enclosure pieces. FIG. 18-1 illustrates the handle 2406 with the enclosure/housing, while FIG. 18-2 illustrates the handle 2406 with a portion of the housing/enclosure removed to reveal the internal features.

As shown in FIG. 18-2, the handle 2406 can include one or more components to route elongate movement members 2410 from the shaft 2404 and attach the elongate movement members 2410 to the manual actuator 2408. For example, the handle 2406 can include a plate structure 2412 with one or more holes/tubes/features to route the elongate movement members 2410 to one or more pulleys 2414. The distal ends of the elongate movement members 2410 can be attached to the pulleys 2414, which are coupled to the manual actuator 2408. The pulleys 2414 can be rotatably supported in the handle 2406 and attached to the manual actuator 2408, such that actuation of the manual actuator 2408 can cause the pulleys 2414 to rotate, thereby pulling (and/or releasing tension of) the elongate movement members 2410. In examples, the pulleys 2414 are coupled to each other through one or more couplers, such as gears, belts, etc. As such, rotation of one pulley 2414 can cause rotation of the other pulley 2414 in the same or a different direction.

The catheter 2402 can be configured to move in a variety of directions, such as at a distal end portion of the shaft 2404. In one illustration, the ends of the elongate movement members 2410 represent two elongate movement members, wherein each elongate movement member 2410 is looped through the distal end portion of the shaft 2404. Here, the distal end portion of the shaft 2404 can be configured to move in two directions, such as up and down or left and right. In another illustration the ends of the elongate movement members 2410 represent proximal ends of four elongate movement members, wherein a distal end of each elongate movement member 2410 is attached to the distal end portion of the shaft 2404. Here, the distal end portion of the shaft 2404 can be configured to move in four directions, such as up, down, left, and right. However, any number of elongate movement members and/or directions of movement can be implemented. In some embodiments, the catheter 2402 includes one or more gears 2416 to facilitate roll of the elongate shaft 2404, such as based on manual input provided via an actuator/control coupled to the one or more gears 2416.

Although the catheter 2402 is illustrated with particular components, other components can be implemented. For example, the plate structure 2412 and/or the pulleys 2414 can be replaced with other components to route the elongate movement members 2410 and/or to facilitate pull/release of the elongate movement members 2410. In some instances, any of the components of other example catheters discussed herein (whether robotic or manual) can alternatively, or additionally, be implemented. Furthermore, any of the components of the catheter 2402 can be implemented in other catheters discussed herein.

As shown in FIG. 19, in some implementations, the handle 2406 of the catheter 2402 is configured to be held/manipulated by a user 2502 in an overhand manner (e.g., a thumbs-up manner). Here, the user's thumb can contact/actuate the manual actuator 2408 and the rest of the user's fingers can grip around the handle 2406 to the opposite side of the handle 2406. The user 2502 can move his/her thumb in an up or down direction (with respect to FIG. 19) to cause the manual actuator 2408 to articulate up and down, resulting in articulation of the distal end portion of the elongate shaft 2404. However, the catheter 2402 can be held/manipulated by a user in a variety of other manners.

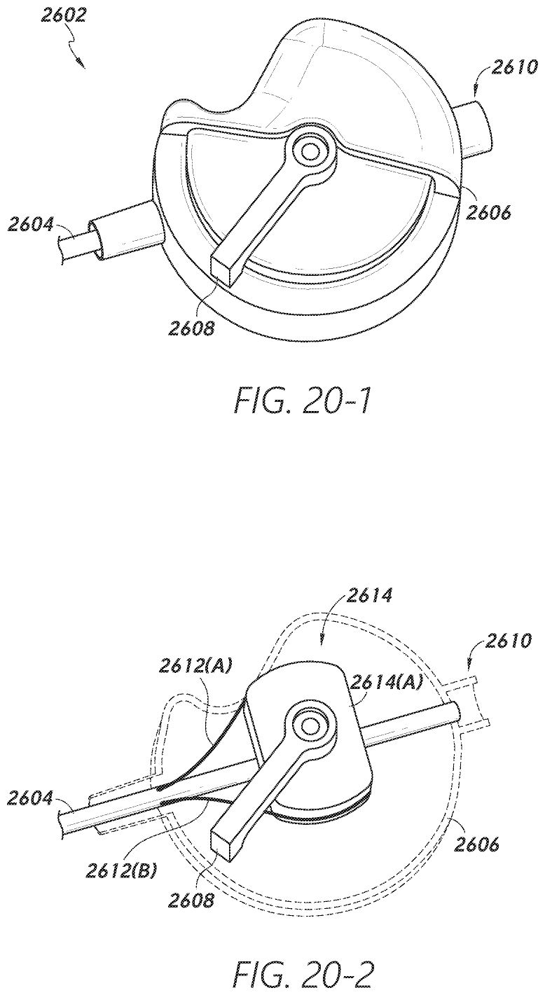

FIGS. 20-1 and 20-2 illustrate another example manually-controllable catheter 2602 in accordance with one or more embodiments of the present disclosure. Here, the catheter 2602 includes a plate structure to facilitate movement of one or more elongate movement members. In particular, the catheter 2602 includes an elongate shaft 2404 coupled to a handle/base 2406 that is configured to control actuation of at least a portion of the elongate shaft 2604. The shaft 2604 can include any of the shafts discussed herein. As shown, the catheter 2602 can include a manual actuator 2608 configured to control actuation of the elongate shaft 2404. The handle 2606 of the catheter 2602 can include one or more housing/enclosure pieces and/or a port 2610 configured to couple to a fluid management system, such as via an aspiration channel/tube. Here, the proximal end of the shaft 2604 is coupled to the port 2610. FIG. 20-1 illustrates the handle 2606 with the enclosure/housing, while FIG. 20-2 illustrates the handle 2606 with a portion of the housing/enclosure removed.

As shown in FIG. 20-2, the manual actuator 2608 is coupled to elongate movement members 2612 via a plate 2614. The elongate movement members 2612 can be coupled to the plate 2412 using an adhesive, fastener, anchors, and so on. In some implementations, the elongate movement members 2612 are attached to a most proximal end 2614(A) of the plate 2614 relative to the port 2610 when the plate 2614 is oriented in the manner shown in FIG. 20-2 (e.g., a default state without articulation of the shaft 2604). As illustrated, the elongate movement members 2612 can exit the shaft 2604 within the handle 2606 and attached to opposite ends of the plate 2614. In use, actuation of the manual actuator 2608 can cause the plate 2614 to rotate within the handle 2606, thereby causing one of the elongate movement members 2612 to be pulled while releasing tension of the other elongate movement member 2612. For example, rotating the manual actuator 2608 toward the proximal end of the shaft 2604 (e.g., in a counterclockwise manner with respect to FIGS. 20-1 and 20-2), can cause the elongate movement number 2612(A) to be released more into the shaft 2604 and cause more of the elongate movement member 2612(B) to be pulled from the shaft 2604.

The catheter 2606 can be configured to move in a variety of directions, such as at a distal end portion of the shaft 2604. In one illustration the ends of the elongate movement members 2612 represent proximal ends of two elongate movement members, wherein a distal end of each elongate movement member 2612 is attached to the distal end portion of the shaft 2604. Here, the distal end portion of the shaft 2604 can be configured to move in two directions, such as up and down or left and right. However, any number of elongate movement members and/or directions of movement can be implemented. In examples, the catheter 2602 is configured to be held in an overhand or underhand manner.

Although the catheter 2602 is illustrated with particular components, other components can be implemented. For example, the plate 2614 and/or other components can be replaced with other components. In some instances, any of the components of other example catheters discussed herein (whether robotic or manual) can alternatively, or additionally, be implemented. Furthermore, any of the components of the catheter 2602 can be implemented in other catheters discussed herein.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is understood with the context as used in general to convey that an item, term, element, etc. may be either X, Y, or Z. Thus, such conjunctive language is not generally intended to imply that certain embodiments require at least one of X, at least one of Y, and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the disclosure herein should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A robotically-controllable catheter assembly comprising:
an elongate shaft including a lumen and configured to couple to an aspiration system to provide aspiration to a target site via the lumen; and
an instrument base coupled to the elongate shaft and configured to control actuation of the elongate shaft, the instrument base including a drive input assembly configured to couple to a drive output assembly associated with a robotic arm.

2. The robotically-controllable catheter assembly of claim 1, wherein the elongate shaft includes another lumen, and the robotically-controllable catheter assembly further comprises:
an elongate movement member slidably disposed in the other lumen and connected to a distal end of the elongate shaft;
wherein the drive input assembly is connected to the elongate movement member to control articulation of the elongate shaft.

3. The robotically-controllable catheter assembly of claim 1, wherein the instrument base includes a port coupled to a proximal end of the elongate shaft and configured to couple to the aspiration system.

4. The robotically-controllable catheter assembly of claim 1, wherein the instrument base includes an identification element associated with an identifier for the robotically-controllable catheter assembly, the identification element including at least one of a radio-frequency identification tag, a Quick Response (QR) code, a bar code, or a magnet.

5. The robotically-controllable catheter assembly of claim 1, further comprising:
a handheld instrument adapter configured to receive manual input to control manipulation of the elongate shaft, the handheld instrument adapter including a coupler configured to couple to the drive input assembly of the instrument base and a manual actuator connected to the coupler and configured to manipulate the coupler.

6. The robotically-controllable catheter assembly of claim 5, wherein the coupler includes a gear assembly engaged with the manual actuator and configured to engage with the drive input assembly.

7. The robotically-controllable catheter assembly of claim 5, further comprising:
a pull wire configured to manipulate the elongate shaft;
wherein the coupler includes a tensioning mechanism configured to disengage the manual actuator from manipulating the drive input assembly and configured to adjust tension of the pull wire.

8. A system comprising:
a base;
a coupler rotatably supported in the base, the coupler configured to couple to a drive input assembly of a robotically-controllable medical instrument; and
a first manual actuator operatively coupled to the coupler and configured to manipulate the coupler to cause the robotically-controllable medical instrument to articulate.

9. The system of claim 8, wherein the coupler includes an engagement assembly coupled to the first manual actuator and configured to couple to the drive input assembly of the robotically-controllable medical instrument.

10. The system of claim 9, wherein the engagement assembly includes (i) a first engagement member to engage with the manual actuator, (ii) a second engagement member configured to engage with the drive input assembly, and (iii) a disengagement mechanism configured to disengage a coupling of the first engagement member to the second engagement member.

11. The system of claim 10, wherein the disengagement mechanism includes a second manual actuator configured to receive manual input to disengage the coupling of the first engagement member to the second engagement member.

12. The system of claim 8, further comprising:
the robotically-controllable medical instrument including (i) an elongate shaft configured to couple to an aspiration system to provide aspiration to a target site, and (ii) an instrument base coupled to the elongate shaft and configured to control actuation of the elongate shaft, the instrument base including the drive input assembly.

13. The system of claim 12, wherein the elongate shaft includes a lumen, and the robotically-controllable medical instrument further includes an elongate movement member slidably disposed in the lumen and connected to a distal end of the elongate shaft;
wherein the drive input assembly is connected to the elongate movement member to control articulation of the elongate shaft.

14. The system of claim 13, wherein the coupler includes a tensioning mechanism configured to disengage the first manual actuator from manipulating the drive input assembly and configured to adjust tension of the elongate movement member.

15. A system comprising:
an elongate shaft including a distal end portion, a proximal end portion, and a lumen, the elongate shaft being configured to couple to an aspiration system to provide aspiration via the lumen; and
a handle coupled to the elongate shaft and configured to operate in:
a robotic mode in which the handle receives robotic input to control articulation of the elongate shaft; and
a manual mode in which the handle receives manual input to control articulation of the elongate shaft.

16. The system of claim 15, further comprising:
a robotic arm including a drive output assembly configured to provide the robotic input to the handle;
wherein the handle is coupled to the drive output assembly of the robotic arm.

17. The system of claim 15, wherein the handle includes a manual actuator coupled to the elongate shaft and configured to receive the manual input.

18. The system of claim 15, wherein the handle includes an instrument base configured to receive the robotic input and an adapter configured to couple to the instrument base, the adapter including a manual actuator configured to receive the manual input.

19. The system of claim 18, wherein the adapter includes a coupler configured to couple to a drive input assembly of the instrument base, the coupler including (i) a first engagement member to engage with the manual actuator, (ii) a second engagement member configured to engage with the drive input assembly, and (iii) a disengagement mechanism configured to disengage a coupling of the first engagement member to the second engagement member.

20. The system of claim 19, wherein the disengagement mechanism includes another manual actuator configured to receive manual input to disengage the coupling of the first engagement member to the second engagement member.
